(19) United States
(12) Patent Application Publication
(10) Pub. No.: US 2024/0247108 A1
(43) Pub. Date: Jul. 25, 2024

(54) POLYOLEFIN MIMIC POLYESTER POLYMERS

(71) Applicant: SABIC Global Technologies B.V., Bergen Op Zoom (NL)

(72) Inventors: Sivadinarayana CHINTA, Bangalore (IN); Alexander STANISLAUS, Bangalore (IN); Girish KORIPELLY, Bangalore (IN); Robbert DUCHATEAU, Bangalore (IN); Kaiwalya SABNIS, Bangalore (IN); Pankaj Singh GAUTAM, Bangalore (IN); Ganesh KANNAN, Bangalore (IN); Ravichander NARAYANASWAMY, Bangalore (IN); Lidia JASINSKA-WALC, Bangalore (IN)

(21) Appl. No.: 18/554,455

(22) PCT Filed: Apr. 8, 2022
(86) PCT No.: PCT/EP2022/059387

§ 371 Date: Oct. 6, 2023

(30) Foreign Application Priority Data
Apr. 8, 2021 (EP) 21167501.2

(51) Int. Cl. C08G 81/02; C07C 29/128; C07C 51/09; C08J 11/24

(52) U.S. Cl. CPC C08G 81/027; C07C 29/1285; C07C 51/09; C08J 11/24; C08J 2367/02

(57) ABSTRACT

A polymer, methods of making the polymer, methods of recycling the polymer and compositions including the polymer are described. The polymer can contain repeating units of Formula (I), where n is 1 and denotes number of repeat units, X is an aliphatic group, and Z is an aliphatic hydro-carbon group having at least 45 carbon atoms, preferably 45 to 1,000 carbon atoms, and has a degree of saturation 98 to 100%. The polymer contains 0.01 to 40 ester groups per 1000 backbone carbon units and has a melt temperature (Tm) of 40° C. to 180° C. as measured by DSC at a heating rate of 10° C. per min.

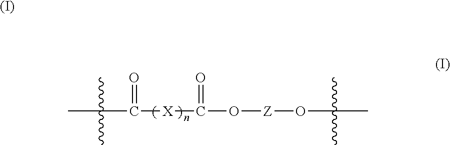

### BACKGROUND OF THE INVENTION

### A. Field of the Invention

The invention generally relates to chemically recyclable polymers.

### B. Description of Related Art

Polyolefins have multiple industrial uses. Polyolefins such as polyethylene and polypropylene constitute the largest volume of synthetic plastic produced worldwide. Polyolefins are used in wide variety of materials, such as films, sheets, foams, fibers, toys, bottles, containers, furniture, electronic parts, and plumbing materials.

An issue with polyolefins is their poor chemical recyclability back to their respective building blocks or monomeric units. For example, the chemical recycling efficiency back to polyolefin building blocks starting from waste plastic is about 40-50%. One reason for this is the chemical recycling process can produce by-products like aromatics, methane, coke, etc. This means full recycling circularity may not be possible to achieve in the current recycling processes with the polymers currently in use.

### SUMMARY OF THE INVENTION

A discovery has been made that provides a solution to at least some of the problems that may be associated with the chemical recyclability of polymers such as polyolefins. In one aspect, the discovery can include providing polyester polymers that have polyolefin like properties (e.g., crystallinity, melt temperature (Tₘ), etc.), that can readily be recycled to their building blocks. This can increase the chemical recycling efficiency when compared with current polyolefin polymers. In one aspect, it was found that polyester polymers, containing less than 40, such as 0.01 to 40 ester groups, per 1,000 backbone carbon atoms, having relatively high degree of saturation, and/or having relatively low degree of branching, can have polyolefin like properties. As, illustrated, in a non-limiting manner in the examples, a polyester polymer according to one example of the present invention can have a melt temperature and crystallinity similar to a polyolefin, and can readily be recycled to the monomers forming the polymer.

One aspect of the present invention is directed to a polymer. The polymer can contain repeating units of Formula I:

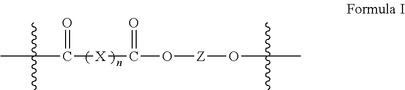

wherein n can be 0 or 1, and denotes number of repeat units and can contain less than 40, such as 0.01 to 40 (e.g., 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or any value or range there between) ester groups per 1,000 backbone carbon atoms.

Z can be an aliphatic group, preferably a aliphatic hydrocarbon group. In some aspects, Z can contain at least 45 carbon atoms, and can have a degree of saturation of 97 to 100%, such as 98 to 100%. In some aspects, Z can contain 45 to 1,000 carbon atoms, such as 50 to 800 carbon atoms, such as 60 to 600 carbon atoms, preferably 100 to 700 carbon atoms. In some aspects, Z can have degree of branching (DB) of 0 to 10%, such as 0 to 9%, such as 0 to 7%. In certain aspects, Z can vary randomly between the repeating units of Formula I. In certain aspects, the number of carbon atoms and/or DB of the Z group, can vary randomly between the repeating units of Formula I. In certain aspects, i) average number of carbon atoms in the Z groups of the polymer can be 45 to 1000, such as 50 to 800, such as 60 to 600, 100 to 700 carbon atoms connected to the oxygen atoms, ii) the Z groups of the polymer can have a polydispersity index of be 1.5 to 4, preferably 1.5 to 3, more preferably 1.5 to 2.5, and/or iii) the average DB of the Z groups of the polymer can be 0 to 10 mol. %, such as 0 to 9 mol. %, such as 0 to 7 mol. %. In certain aspects, Z does not vary between the repeating units of Formula I.

In some aspects, Z can be linear hydrocarbon. In some aspects, Z can be a branched hydrocarbon having a DB of 0.01 to 10%, such as 0.01 to 9%, such as 0.01 to 7%. In some aspects, a Z having at least 45 carbon atoms, preferably 100 to 700 carbon atoms connecting the two oxygen atoms and a degree of branching of 0 to 10%, can provide for ester/backbone carbon atom ratio suitable for obtaining polyolefin like properties. In some aspects, Z can be a polyolefin group. A polyolefin group can be a polyolefin with one H missing at each of the two ends of the polyolefin backbone chain, where the valency of the terminal carbons are satisfied by bonding with the “—O—” groups at the two sides of Z. In some aspects, Z can be a linear polyolefin group. In some aspects, Z can be a branched polyolefin group, having a DB of 0.01 to 10%, such as 0.01 to 9%, such as 0.01 to 7%. In some aspects, Z can contain C₁ to C₁₀ hydrocarbon branches. In some aspects, the polyolefin group can be a polyethylene, poly(ethylene-propylene), or polyethylene-co-α-olefin), such as poly(ethylene-co-1-butene), poly(ethylene-co-1-hexene), or poly(ethylene-co-1-octene) group. In some aspects, Z can be a linear polyethylene group. In some aspects, Z can be a branched polyethylene group containing C₁ to C₁₀ alkyl group branches, and a DB of 0.01 to 10%, such as 0.01 to 9%, such as 0.01 to 7%.

In some aspects, Z can be a poly(α-olefin) group or a poly(α-olefin-co-ethylene) group having a DB greater than 10%, such as 10% to 50%, wherein the α-olefin monomers of the poly(α-olefin) group or poly(α-olefin-co-ethylene) group contain 3 or more carbons. In some aspects, the poly(α-olefin) group can be a polypropylene group, or a polybutylene group, or a poly(propylene-co-ethylene) group. In some aspects, Z can be an atactic, isotactic, or syndiotactic polypropylene group. In some aspects, Z can be random poly(propylene-co-ethylene) group. In certain aspects, Z can be poly(propylene-co-ethylene) group containing 0.7 to 6.6 mol. % of ethylene.

X can be an aliphatic group. X can contain up to 1000 carbon atoms. In some aspects, X can be a linear hydrocarbon. In some aspects, X can be a branched hydrocarbon. In some aspects, X can be a polyolefin group. A polyolefin group of X can be a polyolefin with one H missing at each of the two ends of the polyolefin backbone chain, where the valency of the terminal carbons are satisfied by bonding with the “—COO—” groups at the two sides of X. In some aspects, X can be a linear polyolefin group. In some aspects, X can be a branched polyolefin group having a DB of 0.01 to 50%. In some aspects, X can contain C₁ to C₁₀ hydrocarbon branches. In some aspects, X can be a polyethylene, poly(ethylene-propylene), poly(α-olefin), poly(α-olefin-co-ethylene), or poly(ethylene-co-α-olefin) group. In certain aspects, X can be a poly(ethylene-co-1-butene), polyethylene-co-1-hexene), or poly(ethylene-co-1-octene) group. In some aspects, X can be a polypropylene group, or a polybutylene group, or a poly(propylene-co-ethylene) group. In some aspects, X can be an atactic, isotactic, or syndiotactic polypropylene group. In some aspects, X can be random poly(propylene-co-ethylene) group. In certain aspects, X can vary randomly between the repeating units of Formula I, In certain aspects, i) number of carbon atoms in the X groups can vary randomly between the repeating units of Formula I or ii) the DB of the X groups can vary randomly between the repeating units of Formula I. In certain aspects, X does not vary between the repeating units of Formula I.

In certain aspects, X can contain 45 to 1000 carbon atoms. In certain aspects, X can be a C₁ to C₄₄ aliphatic group. In some particular aspects, X can be a C₁ to C₂₀ aliphatic group, more preferably C₁ to C₁₅, most preferably C₁ to C₈. In some aspects, X can be a linear or branched, and substituted or unsubstituted hydrocarbon. In some aspects, X can have the formula of (1), (2), (3), (4), or (5):

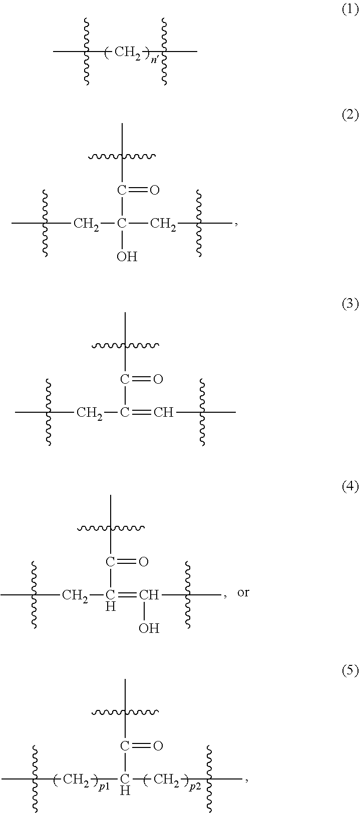

or, any combination thereof
wherein n′ in formula (1) is an integer from 1 to 1000 and denotes number of repeat units, and wherein p1 and p2 in formula (5) can independently be an integer from 0 to 5 and denote number of repeat units. In certain aspects, n′ can be an integer from 1 to 15.

The polymer of the present invention can have a melt temperature (Tₘ) of 40° C. or over. In some aspects, the polymer can have melt temperature (Tₘ) of 40° C. to 170° C., such as 85° C. to 165° C., such as 90° C. to 160° C., such as 95° C. to 150° C., such as 110° C. to 145° C. In some aspects, the number average molecular weight (Mₙ) of the polymer can be 10,000 to 1,000,000 g/mol, preferably of 20,000 to 500,000 g/mol, more preferably of 40,000 to 200,000 g/mol. The Mₙ can be determined as the polyethylene equivalent molecular weight by high temperature size exclusion chromatography performed at 160° C. in trichlorobenzene using polyethylene standards. In some aspects, the polymer can have a polydispersity index (PDI), of 1.5 to 4, preferably 1.8 to 3.

In certain aspects, the polymer can contain repeating units of Formula II:

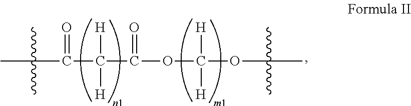

wherein n1 is an integer from 0 to 15 and den number of repeat units, where m1 is an integer from 100 to 700 and denotes the number of repeat units. In some aspects, m1 can be an integer from 200 to 600. In some aspects, m1 can be an integer from 100 to 500. In some aspects, m1 can be an integer from 200 to 500. In some aspects, m1 can be an integer from 300 to 500. In certain aspects, m1 can vary randomly between the repeating units of Formula II, and/or the average of m1s in the polymer can be 100 to 700, such as 200 to 600, such as 300 to 500. In certain aspects, m1 does not vary between the repeating units of Formula II.

In some aspects, the polymer can contain repeating units of Formula III:

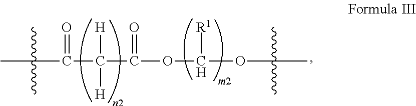

wherein, n2 is an integer from 0 to 15 and denotes number of repeat units, where m2 is integer from 100 to 700 and denotes the number of repeat units. In some aspects, m2 can be an integer from 200 to 600. In some aspects, m2 can be an integer from 300 to 600. In some aspects, m2 can be an integer from 100 to 520. In some aspects, m2 can be an integer from 400 to 520. R¹ can be —H or a C₁ to C₁₀ alkyl group, and varies independently between —H and the C₁ to C₁₀ alkyl group in the repeating units —CHR¹—, wherein DB of —(CHR¹)ₘ₂— group is 0.01 to 10%, e.g. 0.01 to 10% of R¹ is the C₁ to C₁₀ alkyl group, with the rest being —H. In some aspects, n2 can be 2. In some aspects, R¹ can be —H or —CH₂CH₃. In some aspects, the DB of —(CHR¹)ₘ₂— group can be 0.1 to 5%. In certain aspects, m2 can vary randomly between repeating units of Formula III, and/or the average of m2s in the polymer can be 200 to 600, such as 300 to 600, such as 400 to 520. In certain aspects, m2 does not vary between the repeating units of Formula III. In certain aspects, DB of the —(CHR¹)ₘ₂— group can vary randomly between the repeating units of Formula III, and/or the average DB of the —(CHR¹)ₘ₂— groups of the polymer can be 0.01 to 10%. In certain aspects, DB of the —(CHR¹)ₘ₂— group between the repeating units of Formula III does not vary.

In some aspects, the polymer can have Formula IV, and can contain the blocks A and B;

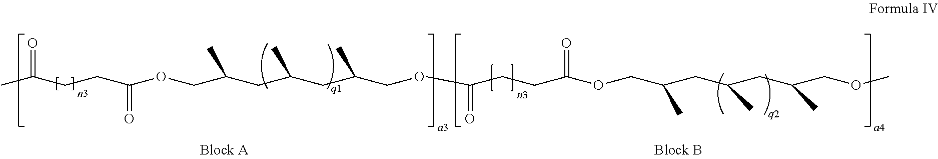

wherein n3 can be an integer from 0 to 14 and denotes number of repeat units, q1 and q2 can independently be integers from 25 to 200, preferably 50 to 125 and denotes number of repeat units, and a3 and a4 are independently an integer. The length of the blocks can be same or different, e.g., a3 and a4 can be same or different. In certain aspects, q1 can vary randomly in the repeating units forming the Block A, and/or in the Block A average of q1s can be 25 to 200, preferably 50 to 125. In certain aspects, q2 can vary randomly in the repeating units forming the Block B, and/or in the Block B average of q2s can be 25 to 200, preferably 50 to 125. In certain aspects, q1 does not vary in the repeating units forming the Block A, and/or, q2 does not vary in the repeating units forming the Block B.

Certain aspects are directed to a method for forming a polymer described herein. The method can include reacting an α,ω-dihydroxy compound having a formula of HO—Z—OH, with i) an acid having a formula of Formula V, ii) an ester of the acid having the formula of Formula V, and/or iii) a cyclic anhydride of the acid having the formula of Formula V. Z can have a structure as described above. n can be 0 or 1 and denotes number of repeat units. The structure of Formula V can be:

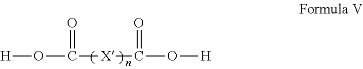

X′ can be an aliphatic group. X′ can contain up to 1000 carbon atoms. In some aspects, X′ can be a linear hydrocarbon. In some aspects, X′ can be a branched hydrocarbon In some aspects, X′ can be a polyolefin group. A polyolefin group of X′ can be a polyolefin with one H missing at each of the two ends of the polyolefin backbone chain, where the valency of the terminal carbons are satisfied by bonding with the “—COO—” groups at the two sides of X′. In some aspects, X′ can be a linear polyolefin group. In some aspects, X′ can be a branched polyolefin group having a DB of 0.01 to 50%. In some aspects, X′ can contain C₁ to C₁₀ hydrocarbon branches. In some aspects, X′ can be a polyethylene, poly(ethylene-propylene), poly(α-olefin), poly(α-olefin-co-ethylene), or poly(ethylene-co-α-olefin) group. In certain aspects, X′ can be a poly(ethylene-co-1-butene), poly(ethylene-co-1-hexene), or poly(ethylene-co-1-octene) group. In some aspects, X′ can be a polypropylene group, or a polybutylene group, or a poly(propylene-co-ethylene) group. In some aspects, X′ can be an atactic, isotactic, or syndiotactic polypropylene group. In some aspects, X′ can be random poly(propylene-co-ethylene) group. In certain aspects, X′ can contain 45 to 1000 carbon atoms. In certain aspects, X′ can be a C₁ to C₄₄ aliphatic group. In some particular aspects, X′ can be a C₁ to C₂₀ aliphatic group. In some aspects, X′ can be a linear or branched, and substituted or unsubstituted hydrocarbon. In some aspects, X′ can have the formula of (1), (6), (7), (8), or (9):

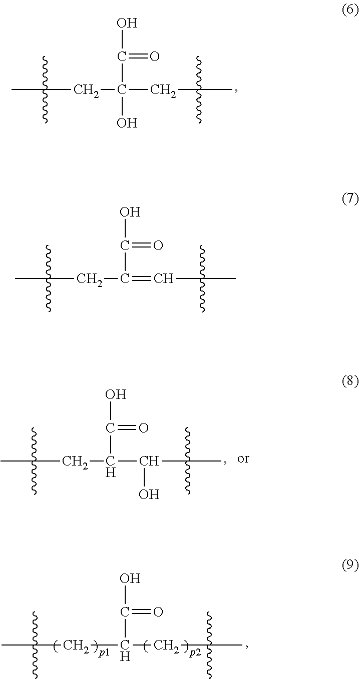

wherein p1 and p2 in formula (9) are independently 0, 1, 2, 3, 4 or 5 and denote number of repeat units. Formula (1) is defined above.

In some aspects, the acid (e.g., of Formula V) can be oxalic acid, malonic acid, succinic acid, maleic acid, glutaric acid, adipic acid, pimelic acid, suberic acid, azelaic acid, sebacic acid, citric acid, aconitic acid, isocytric acid, propane-1,2,3-tricarboxylic acid, pentane-1,3,5-tricarboxylic acid, or any combinations thereof. In some aspects, the ester (e.g. of the acid of Formula V) can be a methyl, ethyl and/or propyl ester. In some aspects, the cyclic anhydride can be malonic anhydride, succinic anhydride, maleic anhydride, glutaric anhydride, adipic anhydride, pimelic anhydride, suberic anhydride, azelaic anhydride, sebacic anhydride, or any combinations thereof.

In some aspects, the α,ω-dihydroxy compound can be reacted with the acid and/or ester and/or cyclic anhydride thereof (e.g., of Formula V) at i) a temperature of 90 to 250° C., and/or ii) under inert atmosphere and/or vacuum.

In some aspects, the acid and/or ester and/or cyclic anhydride thereof (e.g., of Formula V) can be reacted with the α,ω-dihydroxy compound, in presence of a triol, tetraol, and/or polyol (poly>4). The triol, tetraol, and/or polyol can react with the acid and form branches in the polymer. The mol. ratio of i) α,ω-dihydroxy compound, and ii) triol, tetraol, and/or polyol, in the reaction mixture can be 9:1 to 100:1.

Certain aspects are directed to a method for recycling a polymer described herein. The recycling method can include contacting the polymer with water and/or an alcohol under conditions suitable to depolymerize the polymer to produce i) a α,ω-dihydroxy compound having a formula of HO—Z—OH, and ii) an acid having a formula of Formula V, and/or an ester thereof. The polymer can get depolymerized through hydrolysis (e.g., with water) and/or alcoholysis (e.g., with alcohol). In certain aspects, the polymer can be depolymerized by contacting the polymer with methanol to form an α,ω-dihydroxy compound (e.g., HO—Z—OH) and a methyl ester of an acid having a formula of Formula V. In certain aspects, the depolymerization conditions can include a temperature of 100° C. to 250° C. and/or a pressure of 10 barg to 60 barg.

Certain aspects are directed to a first polymer containing repeating units of Formula I, wherein the first polymer is obtained from the polymerization of an α,ω-dihydroxy compound having a formula of HO—Z—OH with an acid (e.g., of formula V), ester and/or cyclic anhydride thereof, and wherein the HO—Z—OH is a recycled HO—Z—OH. The recycled HO—Z—OH can be obtained from depolymerization of a second polymer containing repeating units of Formula I. The first polymer and the second polymer can be chemically the same or different. Acid (or ester thereof) produced during depolymerization of the second polymer can be chemically the same or different than the acid (or ester and/or cyclic anhydride thereof) used during repolymerization of the recycled. HO—Z—OH to form the first polymer.

Certain aspects are directed to a composition containing a polymer described herein. In some aspects, the composition can further contain one or more additional components in addition to the polymer. In some aspects, the composition can be comprised in or in the form of a foam, a fiber, a powder, a film, a layer, or a sheet. Certain aspects are directed to an article of manufacture containing a polymer described herein and/or a composition containing the polymer. The composition and/or article of manufacture can be molded, such as extruded, injection molded, blow molded, compression molded, rotational molded, thermoformed and/or 3-D printed article.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment discussed herein can be implemented with respect to any method or composition of the invention, and vice versa. Furthermore, compositions of the invention can be used to achieve methods of the invention.

The following includes definitions of various terms and phrases used throughout this specification.

The term “degree of branching (DB)” of a group/oligomer/polymer refers to % of branched carbons in the backbone of the group/oligomer/polymer. For example, the following group having the formula of Formula (16), has a degree of branching 25%. The branched carbons in the backbone of the group of Formula 16 is marked with a *. R′ in formula 16 is a branching group, can be an alkyl group, and r is an integer and denotes number of repeat units.

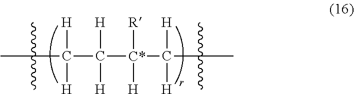

The term “linear hydrocarbon” refers to a hydrocarbon having a continuous carbon chain without side chain branching. The continuous carbon chain may be optionally substituted. The optional substitution can include replacement of at least one hydrogen atom with a functional group, such as hydroxyl, acid, amine, or halogen group; and/or replacement of at least one carbon atom with a heteroatom.

The term “branched hydrocarbon” refers to a hydrocarbon having a linear carbon chain containing branches, such as substituted and/or unsubstituted hydrocarbyl branches, bonded to the linear carbon chain. Optionally, the linear carbon chain can contain additional substitution. Optional additional substitutions can include replacement of at least one carbon atom in the linear carbon chain with a heteroatom and/or replacement of at least one hydrogen atom directly bonded to a carbon atom of the linear chain with a functional group, such hydroxyl, acid, amine, or halogen group.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The polymer of the present invention can “comprise,” “consist(s) essentially of,” or “consist of” particular groups, compositions, etc. disclosed throughout the specification. In one aspect of the present invention, and with reference to the transitional phrase “consist(s) essentially of” or “consisting essentially of,” a basic and novel characteristic of the present invention can include the polymer containing the repeating units of Formula I and/or can have a melt temperature (Tₘ) of 40° C. or higher and/or can be chemically recycled to its building blocks or monomeric units in a relatively efficient manner (e.g., contacted with aqueous and/or alcohol solutions).

All publications mentioned herein are incorporated herein by reference to disclose and describe the methods and/or materials in connection with which the publications are cited.

Other objects, features and advantages of the present invention will become apparent from the following detailed description and examples. It should be understood, however, that the detailed description and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

In the context of the present invention, at least the following 22 aspects are described. Aspect 1 is directed to a polymer comprising repeating units of Formula I:

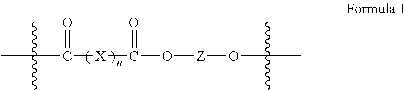

wherein n is 0 or 1 and denotes number of repeat units,
X is an aliphatic group, and
Z is an aliphatic group comprising at least 45 carbon atoms, preferably 45 to 1,000 carbon atoms, and has a degree of saturation of 98 to 100%, and
wherein the polymer has a melt temperature (Tₘ) of 40° C. to 180° C.

Aspect 2 is directed to the polymer of aspect 1, wherein Z is a linear or branched hydrocarbon having a degree of branching (DB) of 0 to 10%.

Aspect 3 is directed to the polymer of any one of aspects 1 to 2, wherein Z is a branched hydrocarbon comprising C₁ to C₂₀ hydrocarbon branches.

Aspect 4 is directed to the polymer of any one of aspects 1 to 3, wherein Z is a polyethylene, poly(ethylene-co-propylene), poly(ethylene-co-1-butene), polyethylene-co-1-hexene), or poly(ethylene-co-1-octene) group.

Aspect 5 is directed to the polymer of aspect 4, wherein Z is a linear or branched polyethylene group.

Aspect 6 is directed to the polymer of aspect 1, wherein Z is polypropylene group, such as an atactic, isotactic, or syndiotactic polypropylene group.

Aspect 7 is directed to the polymer of any one of aspects 1 to 6, wherein X comprises 45 to 1,000 carbon atoms.

Aspect 8 is directed to the polymer of any one of aspects 1 to 6, wherein X is C₁ to C₄₄ aliphatic group, preferably a C₁ to C₂₀ aliphatic group.

Aspect 9 is directed to the polymer of aspect 8, wherein X is selected from

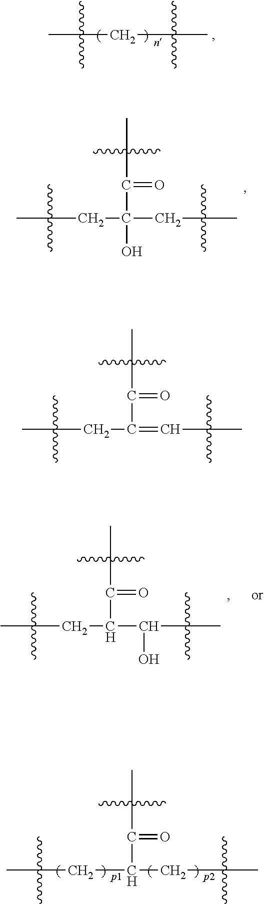

wherein n′ is an integer from 1 to 15, and denotes number of repeat units, and
wherein p1 and p2 is independently 1, 2, or 3, and denote number of repeat units.

Aspect 10 is directed to the polymer of any one of aspects 1 to 9, comprising a number average molecular weight of 10,000 to 1,000,000 g/mol, preferably of 20,000 to 500,000 g/mol, more preferably of 40,000 to 200,000 g/mol, said number average molecular weight being determined as the polyethylene equivalent molecular weight by high temperature size exclusion chromatography performed at 160° C. in trichlorobenzene using polyethylene standards.

Aspect 11 is directed to the polymer of aspect 1, comprising repeating units of Formula II:

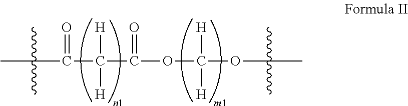

wherein,
n1 is an integer from 0 to 15 and denotes number of repeat units, and
m1 is an integer from 100 to 500 and denotes number of repeat units.

Aspect 12 is directed to the polymer of aspect 1, comprising repeating units of Formula III:

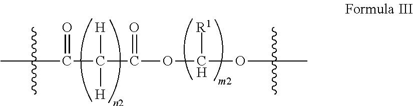

wherein,
n2 is an integer from 0 to 15 and denotes number of repeat units,
m2 is an integer from 100 to 520 and denotes number of repeat units,
R¹ is —H or —CH₂CH₃, and varies independently between H and CH₂CH₃ in the repeating units —CHR¹— and,
—(CHR¹)ₘ₂ group has a DB of 0.1 to 5%.

Aspect 13 is directed to the polymer of aspect 1, comprising the chemical formula of Formula IV

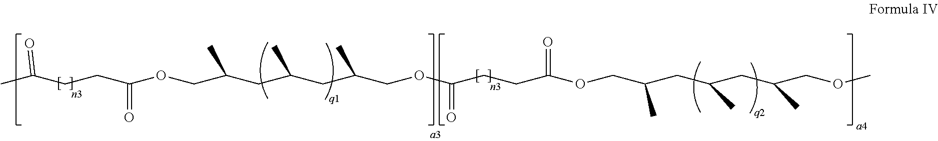

wherein n3 is an integer from 0 to 14 and denotes number of repeat units,
q1 and q2 are independently an integer from 25 to 200 and denote number of repeat units, and
a3 and a4 are independently an integer and denotes number of repeat units.

Aspect 14 is directed to the polymer of aspect 1, comprising repeating units of a first unit having the formula of Formula I, and repeating units of a second unit having the formula of Formula I, wherein X of the first unit has a different chemical formula than the X of the second unit.

Aspect 15 is directed to a method for forming the polymer of any one of aspects 1 to 14, the method comprising:
reacting a α,ω-dihydroxy compound having a formula of HO—Z—OH, with i) an acid having a formula of Formula V, ii) an ester of the acid having the formula of Formula V, and/or iii) a cyclic anhydride of the acid having the formula of Formula V,
wherein Z is an aliphatic group comprising at least 45 carbon atoms, preferably 45 to 1,000 carbon atoms, and has a degree of saturation of 98 to 100%,
wherein Formula V is

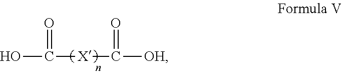

wherein n is 0 or 1 and denotes number of repeat units, and
X′ is an aliphatic group.

Aspect 16 is directed to the method of aspect 15, wherein X′ is selected from

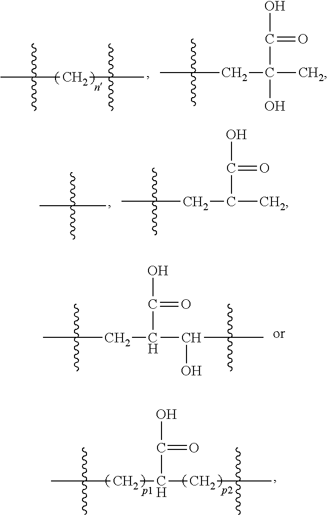

or any combinations thereof,
wherein n′ is an integer denotes number of repeat units, and wherein
p1 and p2 are independently 1, 2, or 3, and denote number of repeat units.

Aspect 17 is directed to the method of aspect 15, wherein the acid is oxalic acid, malonic acid, succinic acid, maleic acid, glutaric acid, adipic acid, pimelic acid, suberic acid, azelaic acid, sebacic acid, citric acid, aconitic acid, isocytric acid, propane-1,2,3-tricarboxylic acid, or pentane-1,3,5-tricarboxylic acid, or any combinations thereof.

Aspect 18 is directed to the method of any one of aspects 14 to 17, wherein the ester is methyl, ethyl and/or propyl ester, and/or wherein the cyclic anhydride is malonic anhydride, succinic anhydride, maleic anhydride, glutaric anhydride, adipic anhydride, pimelic anhydride, suberic anhydride, azelaic anhydride, sebacic anhydride or any combinations thereof.

Aspect 19 is directed to the method of any one of aspects 14 to 18, wherein the α,ω-dihydroxy compound is reacted with the acid or ester or cyclic anhydride thereof at i) a temperature of 90 to 250° C., and/or ii) under inert atmosphere and/or vacuum.

Aspect 20 is directed to a method for recycling a polymer of any one of aspects 1 to 14, the method comprising contacting the polymer with water and/or an alcohol under conditions suitable to depolymerize the polymer through hydrolysis and/or alcoholysis to produce a α,ω-dihydroxy compound having a formula of HO—Z—OH, and an acid having a formula of Formula V, and/or an ester thereof,
wherein Z is an aliphatic group comprising at least 45 carbon atoms, preferably 45 to 1,000 carbon atoms, and has a degree of saturation of 98 to 100%,
wherein Formula V is

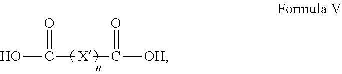

wherein n is 0 or 1 and denotes number of repeat units, and
X′ is an aliphatic group.

Aspect 21 is directed to a composition comprising a polymer of any one of aspects 1 to 14.

Aspect 22 is directed to the composition of aspect 21, wherein the composition is comprised in an article of manufacture.

### BRIEF DESCRIPTION OF THE DRAWINGS

Advantages of the present invention may become apparent to those skilled in the art with the benefit of the following detailed description and upon reference to the accompanying drawings. While the invention is susceptible to various modifications and alternative forms, specific embodiments thereof are shown by way of example in the drawings. The drawings may not be to scale.

FIG. 1 is an X-ray powder diffraction (XRD) inventive polymer (15).

FIG. 2 is a differential scanning calorimetry (DSC) of inventive polymer (15).

FIGS. 3A-3D is an illustration of the polymerization and depolymerization of the present invention. FIG. 3A shows the starting diol. FIG. 3B shows the polymer (15) obtained by polymerization of the diol (14) with succinic acid. FIG. 3C shows a film or sheet made with the polymer (15). FIG. 3D shows the diol (14) obtained after depolymerization of the polymer (15) from the film.

FIG. 4 is a polymerization and depolymerization scheme according to one example of the present invention.

FIG. 5 is a solid state (SS) NMR of a polymer of the present invention (LLDPE mimic).

FIG. 6 shows the ¹H NMR (top) of the inventive polymer in TCE-d2 at 120° C. and compared with α,ω-dihydroxyl hydrogenated polybutadiene (bottom).

FIG. 7 shows DSC thermograms of LLDPE 118NJ, LLDPE-like (inventive) polyester and 80/20 LLDPE/LLDPE-like (inventive polyester) blend. LDPE 2100NO was used additionally as a reference sample.

FIGS. 8A-8C show SAXS patterns of LLDPE, inventive polymer (LLDPE-like material) and LLDPE/inventive polymer blend (80:20).

FIGS. 9 and 10 show storage modulus, loss modulus and loss factor of polyolefin and polyolefin-like materials.

FIG. 11 shows WAXS profiles of pure LLDPE, LLDPE-like polyester inventive polymer and 80/20 LLDPE/LLDPE-like (inventive polymer) blend.

FIG. 12 shows the SSHNMR of HDPE mimic (inventive polymer) and standard HDPE, and succinic acid.

FIG. 13 shows DSC thermograms of HDPE B6246LS, HDPE-mimic polyester (inventive polymer) and 80/20 HDPE/HDPE-mimic (inventive polymer) blend.

FIGS. 14 and 15 show storage modulus, loss modulus and loss factor of polyolefin and polyolefin-like materials.

FIGS. 16A-16C show SAXS patterns of HDPE, inventive polymer blend (HDPE-like material) and 80/20 HDPE/inventive polymer (HDPE-mimic) blend.

FIG. 17 shows WAXS profiles of pure HDPE, HDPE-like (inventive polymer) polyester and 80/20 HDPE/HDPE-mimic (inventive polymer) blend.

FIG. 18 is an XRD of the comparative polymer.

FIG. 19 illustrates melting temperature vs. mol. % of comonomer incorporation of reference polymers (on the line) and polymers of the present invention (above the line).

FIG. 20 illustrates melting temperatures vs. mole fraction of ester groups of comparative polymers (ADMET polymerization process (square monikers) and polyesters from a ring-opening copolymerization process (triangle monikers)), and polyesters of the present invention (round monikers).

### DETAILED DESCRIPTION OF THE INVENTION

A discovery has been made that provides a solution to at least some of the problems associated with chemical recycling of polyolefin polymers. In one aspect, the discovery can include providing a polymer that is more readily recyclable to its chemical building blocks or monomeric units when compared with existing polyolefin polymers such as polyethylene, polypropylene, and/or blends thereof. In one aspect, a polymer of the present invention can have 0.01 to 40 ester groups per 1,000 backbone C atoms and a degree of saturation higher than 97%. As illustrated in a non-limiting manner in the examples, polymers of the current invention can have polyolefin like properties and can readily be recycled to their respective monomeric units.

These and other non-limiting aspects of the present invention are discussed in further detail in the following sections.

### A. Polymer

A polymer of the present invention can contain repeating units of Formula I:

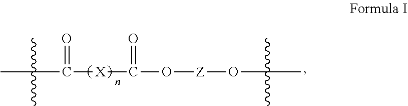

wherein n can be 0 or 1, and denotes number of repeat units. The polymer can contain less than 40, such as 0.01 to 40 (e.g., 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or any value or range there between) ester groups per 1,000 backbone carbon atoms.

Z can be an aliphatic group. Z can contain at least 45 carbon atoms. In certain aspects, Z can vary randomly between the repeating units of Formula I, such as number of carbon atoms and/or DB of the Z groups in the polymer can vary randomly. In certain aspects, Z does not vary between the repeating units of Formula I. In some aspects, Z can contain 45 to 1,000, or equal to any one of, at least any one of, or between any two of 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000 carbon atoms. In some aspects, average of number of carbon atoms in the Z groups of the polymer can be 45 to 1000 or equal to any one of, at least any one of, or between any two of 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000, preferably 100 to 700 carbon atoms connecting the two oxygen atoms.

In some aspects, Z can be a linear hydrocarbon, such as a linear polyolefin group. In some aspects, the linear polyolefin group can have the formula of Formula (10)

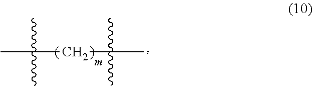 [SMILES: *C*]

where m can be an integer from 45 to 1,000, and denotes number of repeat units. In some aspects, m can be equal to any one of, at least any one of, or between any two of 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000. In some aspects, m can vary randomly between the repeating units of Formula 10, and/or average of m in the polymer, can be 45 to 1,000, or equal to any one of, at least any one of, or between any two of 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000. In some aspects, m does not vary between the repeating units of Formula 10. Formula (10a) is a non-limiting example of a polymer of the present invention, where m (e.g. the Z groups) varies randomly between the repeating units of Formula 10

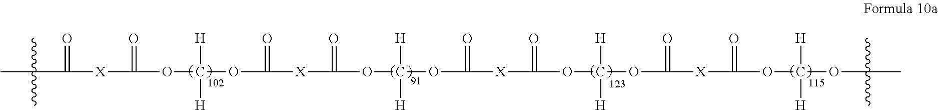

Formula (10b) is a non-limiting example of a polymer of the present invention, where m does not vary between the repeating units of Formula 10

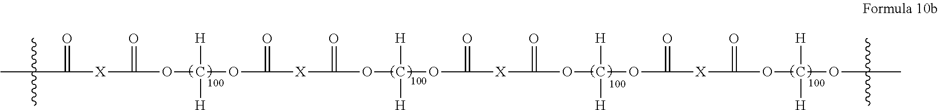

In certain aspects, Z can be a branched hydrocarbon having a degree of branching (DB) of 0.01 to 10%, or equal to any one of, at most any one of, or between any two 0.01, 0.1, 0.5, 1, 2, 3, 4, 5, 6, 7, 8, 9, and 10%. In some aspects, the Z groups in the polymer can have an average DB of 0.01 to 10%, or equal to any one of, at most any one of, or between any two 0.01, 0.1, 0.5, 1, 2, 3, 4, 5, 6, 7, 8, 9, and 10%.

In some aspects, the branched hydrocarbon can contain saturated C₁ to C₁₀ branches (e.g., on the hydrocarbon backbone). In some aspects, the branched hydrocarbon can contain C₁ to C₁₀ alkyl group branches. In some aspects, Z can be a polyolefin having the formula of Formula (11):

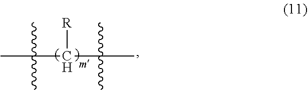

where m′ can be an integer from 45 to 1,000 and denotes number of repeat units, and R can be —H or a C₁ to C₁₀ alkyl group, and varies independently (e.g. between —H and the C₁ to C₁₀ alkyl group) in the repeating units —CHR—, wherein the —(CHR)_{m′}— group has a DB of 0.01 to 10%, or equal to any one of, at most any one of, or between any two of 0.01, 0.1, 1, 2, 3, 4, 5, 6, 7, 8, 9 and 10%. In some aspects, m′ can be equal to any one of, at least any one of, or between any two of 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000. For example, Formula (11a) is a non-limiting example of a polyolefin group with the formula (11), where R is —H or —CH₂CH₃, and R varies independently between —H and the —CH₂CH₃ in the repeating units —CHR—,

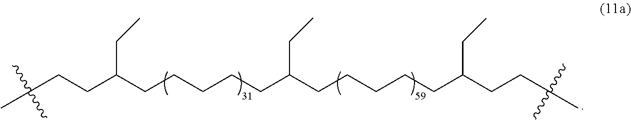

In some aspects, R can be —H or —CH₃. In some aspects, R can be —H or —CH₂CH₃. In some aspects, R can be —H or a C₃ alkyl group. In some aspects, R can be —H or a C₄ alkyl group. In some aspects, R can be —H or a C₅ alkyl group. In some aspects, R can be —H or a C₆ alkyl group. In some aspects, R can be —H or a C₇ alkyl group. In some aspects, R can be —H or a C₈ alkyl group. In some aspects, R can be —H or a C₉ alkyl group. In some aspects, R can be —H or a C₁₀ alkyl group.

In some aspects, m′ can vary randomly between the repeating units of Formula 11, and/or average of m's in the polymer can be, 45 to 1,000, or equal to any one of, at least any one of, or between any two of 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000. In some aspects, m′ does not vary between the repeating units of Formula 11. In some aspects, DB of the —(CHR)_{m′}— groups can vary randomly between the repeating units of Formula 11, and/or average DB of the —(CHR)_{m′}— groups in the polymer can be 0.01 to 10%, or equal to any one of, at most any one of, or between any two of 0.01, 0.1, 1, 2, 3, 4, 5, 6, 7, 8, 9 and 10%. In some aspects, DB of the —(CHR)_{m′}— groups do not vary between the repeating units of Formula 11.

In some aspects, Z can be a polyethylene, poly(ethylene-co-propylene), or poly(ethylene-co-α-olefin) group, having a DB and/or average DB of 0 to 10%, or equal to any one of, at most any one of, or between any two of 0, 0.01, 0.1, 0.5, 1, 2, 3, 4, 5, 6, 7, 8, 9, and 10%. In some aspects, the α-olefin of the poly(ethylene-co-α-olefin) group can be propylene, 1-butene, 4-methyl-1-pentene, 1-hexene, styrene, vinylcyclohexane, 1-octene, norbornene, 5-vinyl-2-norbornene, 5-ethylidene-2-norbornene or 1-decene. In some aspects, the poly(ethylene-co-α-olefin) group can contain less than 5 mol. % of α-olefin. In some aspects, the poly(ethylene-co-α-olefin) group can contain 5 mol. % or more than 5 mol. % of α-olefin. In some aspects, Z can be a linear or branched polyethylene group. The branched polyethylene group can have DB and/or average DB of 0.01 to 10%, or equal to any one of, at most any one of, or between any two 0.01, 0.1, 0.5, 1, 2, 3, 4, 5, 6, 7, 8, 9, and 10%.

In some aspects, Z can be a poly(α-olefin) group or a poly(α-olefin-co-ethylene) group having a DB greater than 10%, such as 10% to 50%, wherein the α-olefin monomers of the poly(α-olefin) group or poly(α-olefin-co-ethylene) group contain 3 or more carbons,. In some aspects, the poly(α-olefin) group can be a polypropylene group, or a polybutylene group, or a poly(propylene-co-ethylene) group. In some aspects, Z can be an atactic, isotactic, or syndiotactic polypropylene group. In some aspects, Z can be random poly(propylene-co-ethylene) group. In certain aspects, Z can be poly(propylene-co-ethylene) group containing 0.7 to 6.6 mol. % of ethylene.

Z can optionally contain one or more side functional groups. In some aspects, the one or more side functional groups can be one or more of hydroxyl, acid, amine, or halogen groups. In some aspects, the functional groups can contain hydrocarbon groups linking the functional group to the hydrocarbon backbone of Z. Z can have a degree of saturation 97 to 100%, or equal to any one of, at most any one of, or between any two 97, 97.5, 98, 98.5, 99, 99.5 and 100%.

In some aspects, n can be 0, and the polymer can contain repeating units of Formula Ia

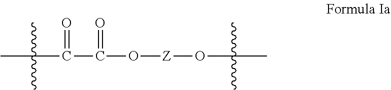

X can be an aliphatic group. X can contain up to 1000 carbon atoms, or equal to any one of, at least any one of, or between any two of 1, 10, 15, 20, 30, 40, 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000 carbon atoms. In certain aspects, X can contain 45 to 1000 carbon atoms. In certain aspects, X can be a C₁ to C₄₄ aliphatic group. In some particular aspects, X can be an aliphatic group containing 1, 2, 3, 4, 5, 6, 7, 8, 9, 10, 11, 12, 13, 14, 15, 16, 17, 18, 19, or 20 carbons. In some aspects, X can be a linear or a branched hydrocarbon. In some aspects, X can be a branched hydrocarbon In some aspects, X can be a polyolefin group. In some aspects, X can be a linear polyolefin group. In some aspects, X can be a branched polyolefin group having a DB of 0.01 to 50%, or equal to any one of, at least any one of, or between any two of 0.01, 0.1, 1, 2, 3, 4, 5, 6, 7, 8, 9, 10, 15, 20, 25, 30, 35, 40, 45, and 50%. In some aspects, X can contain C₁ to C₁₀ hydrocarbon branches. In some aspects, X can be a polyethylene, poly(ethylene-propylene), poly(α-olefin), poly(α-olefin-co-ethylene), or poly(ethylene-co-α-olefin) group. In certain aspects, X can be a poly(ethylene-co-1-butene), poly(ethylene-co-1-hexene), or poly(ethylene-co-1-octene) group. In some aspects, X can be a polypropylene group, or a polybutylene group, or a poly(propylene-co-ethylene) group. In some aspects, X can be an atactic, isotactic, or syndiotactic polypropylene group. In some aspects, X can be random poly(propylene-co-ethylene) group. In some aspects, the one or more side functional groups of X can be one or more of oxy, hydroxyl, acid, amine, or halogen groups. In some aspects, the functional groups can contain hydrocarbon groups linking the functional group to the backbone of X. In certain aspects, X can vary randomly between the repeating units of Formula I. In certain aspects, i) number of carbon atoms in the X groups can vary randomly between the repeating units of Formula I or iii) the DB of the X groups can vary randomly between the repeating units of Formula I. In some aspects, average of number of carbon atoms in the X groups of the polymer can be 1 to 1000 or equal to any one of, at least any one of, or between any two of 1, 2, 3, 4, 5, 6, 7, 8, 9, 10, 11, 12, 13, 14, 15, 16, 17, 18, 19, 20, 25, 30, 35, 40, 44, 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000. In some aspects, the X groups in the polymer can have an average DB of 0.01 to 50%, or equal to any one of, at most any one of, or between any two 0.01, 0.1, 1, 2, 3, 4, 5, 6, 7, 8, 9, 10, 15, 20, 25, 30, 35, 40, 45, and 50%. In certain aspects, X does not vary between the repeating units of Formula I.

In some aspects, n can be 1, X can have the formula of Formula (1), and n′ can be and/or average of n′ in the polymer can be 1 to 1000, or equal to any one of, at least any one of, or between any two of 1, 10, 15, 20, 30, 40, 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000.

In some aspects, n can be 1, X can have the formula of Formula (I), and the polymer can contain repeating units of Formula Ib:

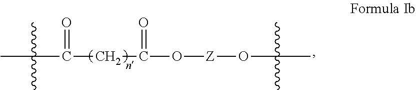

wherein n′ can be 1, 2, 3, 4, 5, 6, 7, 8, 9, 10, 11, 12, 13, 14 or 15, and denotes number of repeat units.

In some aspects, n can be 1, X can have the formula of Formula (3), and the polymer can contain repeating units of Formula Ic

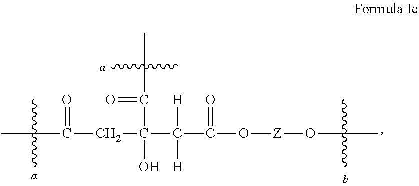

wherein the units (Formula Ic) are bonded through bonding between “a” and “b” ends.

In some aspects, n can be 1, X can have the formula of Formula (3), and the can contain repeating units of Formula Id:

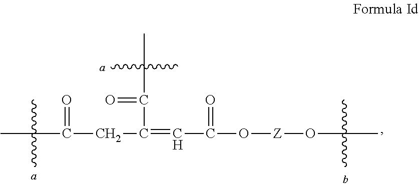

wherein the units (Formula Id) are bonded through bonding between “a” and “b” ends.

In some aspects, n can be 1, X can have the formula of Formula (4), and the polymer can contain repeating units of Formula Ie:

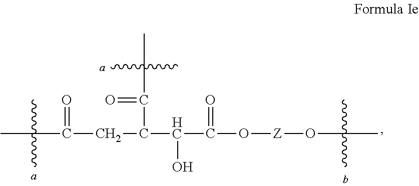

wherein the units (Formula Ie) are bonded through bonding between “a” and “b” ends.

In some aspects, n can be 1, X can have the formula of Formula (5), and the polymer can contain repeating units of Formula If:

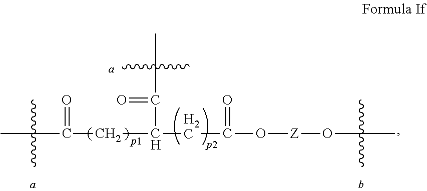

wherein the units (Formula If) are bonded through bonding between “a” and “b” ends.

Formula (1) to (5) are described above.

In certain aspects, the polymer can contain i) repeating units of a first unit having the formula of Formula I, and ii) repeating units of a second unit having the formula of Formula I, wherein X of the first unit can have a different formula than the X of the second unit. In certain aspects, X of the first unit can be a linear hydrocarbon, and the X of the second unit can contain one or more side functional groups, such as oxy groups. The second unit can introduce branching in polymer. The second unit can be bound to three or more monomers. In some aspects, X of the first unit has the chemical formula of Formula (1), and X of the second unit has the chemical formula of Formula (2), (3), (4) or (5). The Z of the first unit and the second unit can be same or different, e.g. can have same or different chemical formula. In some aspects, Z of the first unit and the second unit can have the same formula. In some aspects, the polymer can contain the first units and the second units arranged in blocks, randomly or in alternate. In some aspects, the first units and the second units can be arranged randomly in the polymer. In certain aspects, the ratio of mol. % of the first unit and second unit in the polymer can be 9:1 to 999:1, or equal to any one of, at least any one of, or between any two of 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 200:1, 300:1, 400:1, 500:1, 600:1, 700:1, 800:1, 900:1, and 999:1.

The melt temperature (Tₘ) of the polymer can be equal to or greater than 40° C. In some aspects, Tₘ of the polymer can be 40° C. to 180° C., or equal to any one of, at least any one of, or between any two of 40, 45, 50, 55, 60, 65, 70, 75, 80, 85, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 175 and 180° C. The Tₘ of the polymer can be measured by differential scanning calorimetry performed at a heating rate of 10° C. per minute and wherein the melting temperature corresponds to the melting peak in a second run. In some aspects, the number average molecular weight (Mₙ) of the polymer can be 10,000 to 1,000,000 g/mol, or equal to any one of, at least any one of, or between any two of 10,000; 20,000; 40,000; 50,000; 60,000; 70,000; 80,000; 90,000; 100,000; 110,000; 120,000; 130,000; 140,000; 150,000; 160,000; 170,000; 180,000; 190,000; 200,000; 250,000; 300,000; 350,000; 400,000; 450,000; 500,000; 550,000; 600,000; 650,000; 700,000; 800,000; 900,000; and 1,000,000 g/mol, as determined as the polyethylene equivalent molecular weight by high temperature size exclusion chromatography performed at 160° C. in trichlorobenzene using polyethylene standards. In some aspects, the polymer can have a polydispersity index (PDI), of 1 to 4.0, or equal to any one of, at least any one of, or between any two of 1. 1.1, 1.2, 1.3, 1.4, 1.5, 1.6, 1.8, 2, 2.2, 2.4, 2.6, 2.8, 3, 3.2, 3.4, 3.6, 3.8, and 4.

In certain aspects, the polymer can contain repeating units of Formula II:

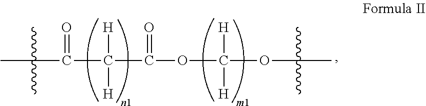

wherein n1 is 0, 1, 2, 3, 4, 5, 6, 7, 8, 9, 10, 11, 12, 13, 14 or 15, and denotes number of repeat units and wherein m1 is an integer from 100 to 700, or equal to any one of, at least any one of, or between any two of 100, 150, 200, 250, 300, 350, 400, 450, 500, 550, 600, 650 and 700, and denotes number of repeat units. In certain aspects, m1 can vary randomly between the repeating units of Formula II, and/or average of m1s of the polymer can be 100 to 700, or equal to any one of, at least any one of, or between any two of 100, 150, 200, 250, 300, 350, 400, 450, 500, 550, 600, 650 and 700. In certain aspects, m1 does not vary between the repeating units of Formula II.

In some aspects, the polymer can have repeating units of Formula III:

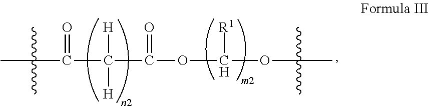

wherein n2 is 0, 1, 2, 3, 4, 5, 6, 7, 8, 9, 10, 11, 12, 13, 14 or 15, and denotes number of repeat units, and wherein m2 is an integer from 100 to 700, or equal to any one of, at least any one of, or between any two of 100, 150, 200, 250, 300, 350, 400, 450, 500, 520, 550, 600, 650 and 700, and denotes number of repeat units. R¹ can be —H or a C₁ to C₁₀ alkyl group, and varies independently (e.g. between —H and the C₁ to C₁₀ alkyl group) in the repeating units —CHR¹—, wherein the —(CHR¹)ₘ₂— group has a DB of 0.01 to 10%, or equal to any one of, at most any one of, or between any two of 0.01, 0.1, 1, 2, 3, 4, 5, 6, 7, 8, 9 and 10%. In some aspects, R¹ can be —H or —CH₃. In some aspects, R¹ can be —H or —CH₂CH₃. In some aspects, R¹ can be —H or a C₃ alkyl group. In some aspects, R¹ can be —H or a C₄ alkyl group. In some aspects, R¹ can be —H or a C₅ alkyl group. In some aspects, R¹ can be —H or a C₆ alkyl group. In some aspects, R¹ can be —H or a C₇ alkyl group. In some aspects, R¹ can be —H or a C₈ alkyl group. In some aspects, R¹ can be —H or a C₉ alkyl group. In some aspects, R¹ can be —H or a C₁₀ alkyl group. In certain aspects, m2 can vary randomly between the repeating units of Formula III, and/or the average of m2s in the polymer can be 200 to 600, or equal to any one of, at least any one of, or between any two of 100, 150, 200, 250, 300, 350, 400, 450, 500, 520, 550, 600, 650 and 700. In certain aspects, m2 does not vary between the repeating units of Formula III. In certain aspects, DB of the —(CHR¹)ₘ₂— groups can vary randomly between the repeating units of Formula III, and/or the average DB of the —(CHR¹)ₘ₂— groups in the polymer can be 0.01 to 10%, or equal to any one of, at most any one of, or between any two of 0.01, 0.1, 1, 2, 3, 4, 5, 6, 7, 8, 9 and 10%. In certain aspects, DB of the —(CHR¹)ₘ₂— groups between the repeating units of Formula III does not vary.

In certain particular aspects, i) n2 can be 2, ii) R¹ can be —H or —CH₂CH₃, varies independently (e.g. between —H and —CH₂CH₃) in the repeating units —CHR¹—, and the —(CHR¹)ₘ₂— group has a DB of 0.1 to 5%, iii) m2 can be 400 to 520, iv) Mₙ of the polymer can be 90,000 to 120,000 g/mol, v) Tₘ of the polymer can be ranging from 90° C. to 110° C., or any combinations thereof. In some particular aspects, i) n2 can be 2, ii) R¹ can be —H or —CH₂CH₃, varies independently (e.g. between —H and —CH₂CH₃) in the repeating units —CHR¹—, and the —(CHR¹)ₘ₂— group has a DB of 0.1 to 5%, iii) m2 can be 400 to 520, iv) Mₙ of the polymer can be 90,000 to 120,000 g/mol, and v) Tₘ of the polymer can be ranging from 90° C. to 110° C.

In some aspects, the polymer can have repeating units of Formula III and Formula IV, wherein the units are bonded through bonding between “a” and “b” ends:

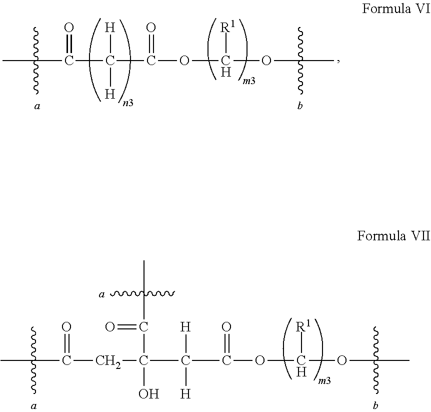

wherein n3 is 0, 1, 2, 3, 4, 5, 6, 7, 8, 9, 10, 11, 12, 13, 14 or 15, and denotes number of repeat units, wherein m3 in Formula VI and VII can independently be an integer from 100 to 700, or equal to any one of, at least any one of, or between any two of 100, 150, 200, 250, 300, 350, 400, 450, 500, 520, 550, 600, 650 and 700, and denotes number of repeat units. R¹ in Formula VI and VII can independently be —H or a C₁ to C₁₀ alkyl group, and varies independently (e.g. between —H and the C₁ to C₁₀ alkyl group) in the repeating units —CHR¹—, wherein the —(CHR¹)ₘ₃— groups in Formula VI and VII can independently have a DB of 0.01 to 10%, or equal to any one of, at most any one of, or between any two of 0.01, 0.1, 1, 2, 3, 4, 5, 6, 7, 8, 9 and 10%. In some aspects, R¹ can be —H or —CH₃. In some aspects, R¹ can be —H or —CH₂CH₃. In some aspects, R¹ can be —H or a C₃ alkyl group. In some aspects, R¹ can be —H or a C₄ alkyl group. In some aspects, R¹ can be —H or a C₅ alkyl group. In some aspects, R¹ can be —H or a C₆ alkyl group. In some aspects, R¹ can be —H or a C₇ alkyl group. In some aspects, R¹ can be —H or a C₈ alkyl group. In some aspects, can be —H or a C₉ alkyl group. In some aspects, R¹ can be —H or a C₁₀ alkyl group. In certain aspects, m3 can vary randomly between the repeating units of Formula VI. In certain aspects, m3 can vary randomly between the repeating units of Formula VII. In certain aspects, m3 does not vary between the repeating units of Formula VI. In certain aspects, m3 does not vary between the repeating units of Formula VII. In certain aspects, DB of the —(CHR¹)ₘ₃— groups can vary randomly between the repeating units of Formula VI. In certain aspects, DB of the —(CHR¹)ₘ₃— groups can vary randomly between the repeating units of Formula VII. In certain aspects, DB of the —(CHR¹)ₘ₃— groups do not vary between the repeating units of Formula VI. In certain aspects, m3 does not vary between the repeating units of Formula VII. The units of Formula VI and Formula VII can be randomly located in the polymer, and can have mol. ratio of 9:1 to 999:1.

### B. Method of Forming the Polymer

Certain aspects are directed to a method for forming a polymer described herein. The method can include reacting a α,ω-dihydroxy compound having a formula of HO—Z—OH, with an i) acid having a formula of Formula V, ii) an ester of the acid having the formula of Formula V, and/or iii) an cyclic anhydride of the acid having the formula of Formula V,

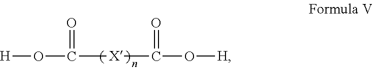

wherein n is 0 or 1 and denotes number of repeat units, and
Z is as described above.

X′ can be an aliphatic group. X′ can and/or on average contain up to 1000 carbon atoms, or equal to any one of, at most any one of, or between any two of 1, 10, 15, 20, 30, 40, 45, 50, 55, 60, 65, 70, 80, 90, 100, 150, 200, 220, 240, 260, 280, 300, 320, 340, 360, 380, 400, 420, 440, 460, 480, 500, 520, 540, 560, 580, 600, 650, 700, 750, 800, 850, 900, 950, and 1,000 carbon atoms. In certain aspects, X′ can contain 45 to 1000 carbon atoms. In certain aspects, X can be a C₁ to C₄₄ aliphatic group. In some particular aspects, X′ can be an aliphatic group containing 1, 2, 3, 4, 5, 6, 7, 8, 9, 10, 11, 12, 13, 14, 15, 16, 17, 18, 19, or 20 carbons. In some aspects, X′ can be a linear or a branched hydrocarbon. In some aspects, X′ can be a branched hydrocarbon having a DB of, and/or an average DB of 0.01 to 50%, or equal to any one of, at least any one of, or between any two of 0.01, 0.1, 1, 2, 3, 4, 5, 6, 7, 8, 9, 10, 15, 20, 25, 30, 35, 40, 45, and 50%. In some aspects, X′ can be a polyolefin group. In some aspects, X′ can be a linear polyolefin group. In some aspects, X′ can be a branched polyolefin group. In some aspects, X can contain C₁ to C₁₀ hydrocarbon branches. In some aspects, X′ can be a polyethylene, poly(ethylene-propylene), poly(α-olefin), poly(α-olefin-co-ethylene), or poly(ethylene-co-α-olefin) group. In certain aspects, X can be a poly(ethylene-co-1-butene), poly(ethylene-co-1-hexene), or poly(ethylene-co-1-octene) group. In some aspects, X′ can be a polypropylene group, or a polybutylene group, or a poly(propylene-co-ethylene) group. In some aspects, X′ can be an atactic, isotactic, or syndiotactic polypropylene group. In some aspects, X′ can be random poly(propylene-co-ethylene) group. In some aspects, X′ can contain one or more side functional groups. In some aspects, the one or more side functional groups can be one or more of oxy, hydroxyl, acid, amine, or halogen groups. In some aspects, the functional groups can contain hydrocarbon groups linking the functional group to the backbone of X′. In some aspects, X′ can have the formula of formula (1), (6), (7), (8), or (9) or any combination thereof. In some aspects, a combination of acids, with different X′ can be used. In some aspects, acids with different X′ can be used, providing a polymer where X varies, such as carbon atoms and/or DB of X varies, randomly between the repeating units of Formula I. In some aspects, the acid (e.g., of Formula V) can be oxalic acid, malonic acid, succinic acid, maleic acid, glutaric acid, adipic acid, pimelic acid, suberic acid, azelaic acid, sebacic acid, citric acid, aconitic acid, isocytric acid, propane-1,2,3-tricarboxylic acid, pentane-1,3,5-tricarboxylic acid, or any combinations thereof. In some aspects, the ester (e.g. of the acid having the formula of Formula V) can be methyl, ethyl and/or propyl ester. In some aspects, the cyclic anhydride can be malonic anhydride, succinic anhydride, maleic anhydride, glutaric anhydride, adipic anhydride, pimelic anhydride, suberic anhydride, azelaic anhydride, sebacic anhydride or any combinations thereof.

In some aspects, the HO—Z—OH can be reacted with the acid (e.g., of Formula V) or ester and/or cyclic anhydride thereof at i) a temperature of 90 to 250° C., or equal to any one of, at least any one of, or between any two of 90, 100, 110, 120, 130, 140, 150, 160, 170, 180, 190, 200, 210, 220, 230, 240, and 250° C. and/or ii) under inert atmosphere and/or vacuum. In some aspects, the reaction can include esterification at 90 to 250° C., and/or under inert atmosphere, followed by polycondensation at 90 to 250° C., and/or under vacuum, e.g. at pressure below 0.5 mbarg, such as below 0.1 mbarg, such as around 0.05 mbarg. The HO—Z—OH can be reacted with the acid, ester and/or cyclic anhydride (e.g., of the acid of Formula V) at a mole ratio of 5:95 to 95:5, or equal to any one of, at least any one of, or between any two of, 5:95, 10:90, 15:85, 20:80, 25:75, 30:70, 35:65, 40:60, 45:55, 50:50, 55:45, 60,:40, 65:35, 70:30, 75:25, 80:20, 85:15, 90:10, and 95:05.

In some aspects, the method can include reacting the α,ω-dihydroxy compound HO—Z—OH with i) a first acid having the formula of Formula V (and/or an ester, and/or cyclic anhydride thereof), and ii) a second acid having the formula of Formula V (and/or an ester, and/or cyclic anhydride thereof), wherein X′ of the Formula V of the first acid is different than the X′ of the Formula V of the second acid. In some aspects, the X′ of the Formula V of the first acid can be a linear hydrocarbon, and the X′ of the Formula V of the second acid can contain one or more side functional groups. In some aspects, X′ of the Formula V of the first acid has the formula of formula (1), and X′ of the Formula V of the second acid has the formula of formula (6), (7), (8), or (9). In some aspects, the first acid can be oxalic acid, malonic acid, succinic acid, maleic acid, glutaric acid, adipic acid, pimelic acid, suberic acid, azelaic acid, sebacic acid, or any combinations thereof. In some aspects, the second acid can be citric acid, aconitic acid, isocytric acid, propane-1,2,3-tricarboxylic acid, pentane-1,3,5-tricarboxylic acid, or any combinations thereof. In certain aspects, the compound HO—Z—OH can be polymerized with more than two acids selected from oxalic acid, malonic acid, succinic acid, maleic acid, glutaric acid, adipic acid, pimelic acid, suberic acid, azelaic acid, sebacic acid, citric acid, aconitic acid, isocytric acid, propane-1,2,3-tricarboxylic acid, and pentane-1,3,5-tricarboxylic acid, and/or esters, and/or anhydride thereof.

In some aspects, the α,ω-dihydroxy compound HO—Z—OH can be reacted with the a) first acid and/or ester and/or cyclic anhydride thereof, and b) the second acid and/or ester and/or cyclic anhydride thereof, at i) a temperature of 90 to 250° C., or equal to any one of, at least any one of, or between any two of 90, 100, 110, 120, 130, 140, 150, 160, 170, 180, 190, 200, 210, 220, 230, 240, and 250° C. and/or ii) under inert atmosphere and/or vacuum. In some aspects, the reaction (e.g. of HO—Z—OH with the first acid and/or ester and/or cyclic anhydride thereof, and the second acid and/or ester and/or cyclic anhydride thereof,) can include esterification at 90 to 250° C., and/or under inert atmosphere, followed by polycondensation at 90 to 250° C., and/or under vacuum, e.g. at pressure below 0.5 mbarg, such as below 0.1 mbarg, such as around 0.05 mbarg. The HO—Z—OH can be reacted with the first acid, ester and/or cyclic anhydride thereof at a mole ratio of 5:95 to 95:5, or equal to any one of, at least any one of, or between any two of, 5:95, 10:90, 15:85, 20:80, 25:75, 30:70, 35:65, 40:60, 45:55, 50:50, 55:45, 60,:40, 65:35, 70:30, 75:25, 80:20, 85:15, 90:10, and 95:05. The first acid and the second acid can be reacted with the HO—Z—OH at a first acid: second acid mole ratio of 9:1 to 999:1, or equal to any one of, at least any one of, or between any two of 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 200:1, 300:1, 400:1, 500:1, 600:1, 700:1, 800:1, 900:1, and 999:1.

In certain aspects, the reaction, (e.g., esterification and/or polycondensation of HO—Z—OH with the acid and/or ester and/or cyclic anhydride thereof, or of HO—Z—OH with the first acid and/or ester and/or cyclic anhydride thereof, and the second acid and/or ester and/or cyclic anhydride thereof) can be performed in presence of a catalyst. In some aspects, catalyst used can include but are not limited to a mineral acid, organic acid, organic base, and/or metallic compound. In some aspects, the metallic compound can be a hydrocarbyl, oxide, chloride, carboxylate, alkoxide, aryloxide, amide, salen complex, β-ketiminato complex, or guanidinato complex, of a metal. In some aspects, the metal can be Li, Na, K, Mg, Ca, Sc, Y, lanthanides, Ti, Zr, Zn, Mo, Mn, Al, Ga, Bi, Sb, or Sn. In some aspects, the catalyst can be Ti(OiPr)4, Ti(OBu)4, Al(OiPr)3, Sn(2-ethyl-hexanoate)2, MoO3, or any combinations thereof. In certain aspects, a combination of catalyst can be used.

In some aspects, the acid and/or ester and/or cyclic anhydride thereof (e.g., of Formula V) can be reacted with the α,ω-dihydroxy compound, in presence of a triol, tetraol, and/or polyol (poly>4). The triol, tetraol, and/or polyol can react with the acid and form branches in the polymer. The mol. ratio of i) α,ω-dihydroxy compound, and ii) triol, tetraol, and/or polyol, in the reaction mixture can be be 9:1 to 100:1 or equal to any one of, at least any one of, or between any two of 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, and 100:1. In some aspects, the triol or tetraol can be glycerol, trimethalolmethane, trimethalolethane, trimethalolpropane, 3-hydroxymethyl-1,5-pentanediol, pentaerythritol, or any combinations thereof.

### C. Method of Recycling the Polymer

Certain aspects, are directed to a method for recycling a polymer described herein. The recycling can include, depolymerizing the polymer. The polymer can be depolymerized to obtain a α,ω-dihydroxy compound having a formula of HO—Z—OH. In certain aspects, the depolymerization method can include hydrolysis and/or alcoholysis of the polymer to obtain the compound of formula HO—Z—OH, and the acid of Formula V (e.g., via hydrolysis), and/or an ester of the acid of Formula V (e.g., via alcoholysis). In certain aspects, the depolymerization of the polymer can produce i) the compound HO—Z—OH, ii) a first acid having a formula of Formula V (e.g., via hydrolysis), and/or an ester thereof (e.g., via alcoholysis), and iii) a second acid having the formula of Formula V (e.g., via hydrolysis) and/or an ester thereof (e.g., via alcoholysis), wherein X′ of the Formula V of the first acid is different than the X′ of the Formula V of the second acid. In some aspects, the X′ of the Formula V of the first acid can be a linear hydrocarbon, and the X′ of the Formula V of the second acid can contain one or more side functional groups. In some aspects, X′ of the Formula V of the first acid has the formula of formula (1), and X′ of the second acid has the formula of formula (6), (7), (8), or (9). In some aspects, the first acid can be oxalic acid, malonic acid, succinic acid, maleic acid, glutaric acid, adipic acid, pimelic acid, suberic acid, azelaic acid, sebacic acid, or any combinations thereof. In some aspects, the second acid can be citric acid, aconitic acid, isocytric acid, propane-1,2,3-tricarboxylic acid, pentane-1,3,5-tricarboxylic acid, or any combinations thereof. In certain aspects, the depolymerization method can include methanolysis of the polymer under conditions suitable to obtain an compound of formula HO—Z—OH, and a methyl ester of an acid of Formula V (or methyl esters of the first and second acids). In some aspects, the methanolysis conditions can include i) a temperature of 100° C. to 250° C., or equal to any one of, at least any one of, or between any two of 100, 110, 120, 130, 140, 150, 160, 170, 180, 190, 200, 210, 220, 230, 240, and 250° C. and/or ii) a pressure of 10 barg to 60 barg, or equal to any one of, at least any one of, or between any two of 10, 15, 20, 25, 30, 35, 40, 45, 55 and 60 barg. In some aspects, the depolymerization can be performed at an inert atmosphere. Catalyst used for depolymerization, such as methanolysis can include a mineral acid, organic acid, organic base, and/or metallic compound. In some aspects, the metallic compound can be a hydrocarbyl, oxide, chloride, carboxylate, alkoxide, aryloxide, amide, salen complex, β-ketiminato complex, or guanidinato complex, of a metal. In some aspects, the metal can be Li, Na, K, Mg, Ca, Sc, Y, lanthanides, Ti, Zr, Zn, Mo, Mn, Al, Ga, Bi, Sb, or Sn. In some aspects, the catalyst can be Ti(OiPr)4, Ti(OBu)4, Al(OiPr)3, Sn(2-ethyl-hexanoate)2, MoO3, or any combinations thereof.

In certain aspects, the method of recycling can include repolymerization of the recycled HO—Z—OH, e.g., obtained from the depolymerization process. The recycled HO—Z—OH can be repolymerized to form a polymer described herein. In some aspects, the recycled HO—Z—OH can be repolymerized with an acid having the formula of Formula V, an ester, and/or cyclic anhydride thereof (e.g. of the acid of Formula V). In some aspects, the recycled HO—Z—OH can be repolymerized with i) a first acid having the formula of Formula V (and/or an ester, and/or cyclic anhydride thereof) ii) a second acid having the formula of Formula V (and/or an ester, and/or cyclic anhydride thereof), wherein X′ of the Formula V of the first acid is different than the X′ of the Formula V of the second acid.

### D. Compositions and Article of Manufacture Containing the Polymer

The polymers described herein can be included in a composition. In some aspects, the composition can contain a blend of the polymer (e.g., containing repeating units of formula I) and one or more other polymers. In some aspects, the one or more other polymers can be polyethylene, polypropylene, polystyrene, polyethylene terephthalate, polybutylene terephthalate, polybutylene succinate, polybutylene adipate, polyvinyl acetate, ethyl vinyl alcohol, poly(methyl acrylate), poly(methyl methacrylate), polypropylene carbonate, bisphenol A polycarbonate, polysulphonate, polyurethanes, polyamides, synthetic rubber, mineral oils, or any combinations thereof. In some aspects, the composition can further include one or more additives. The one or more additives may include, but are not limited to, a scratch-resistance agent, an antioxidant, a flame retardant, an UV absorber, a photochemical stabilizer, a filler such as glass and/or mineral filler, an optical brightener, a surfactant, a processing aid, a mold release agent, a pigment, flow modifiers, foaming agents or any combinations thereof. In some aspects, the compositions can be comprised in or in the form of a foam, a film, a layer, a sheet, a molded article, a welded article, a filament, a fiber, a wire, a cable, or a powder. In one example, the composition is incorporated into a film. Specifically, the film may include at least one film layer that includes the composition. In further aspects the film includes at least a second film layer.

Certain aspects are directed to an article of manufacture containing a polymer described herein and/or a composition containing the polymer. The composition and/or article of manufacture can be molded, such as extruded, injection molded, blow molded, compression molded, rotational molded, thermoformed and/or 3-D printed article. In some aspects, the article of manufacture can be a personal equipment part, an automobile part, plumbing material, construction material, a consumer electronics housing, a personal equipment part, a kitchen appliance, furniture, or a home appliance component.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Instrumentation

Thermal analysis was carried out on a DSC Q100 from TA Instruments at a heating rate of 10° C. per minute. First and second runs were recorded after cooling down to about −40° C. The melting temperatures reported correspond to second runs.

Dynamic Mechanical Thermal Analysis (DMTA) was performed using TA Instruments Q800 apparatus. Analysis required compression molded bars, which dimensions were: 50 mm×10 mm×0.5 mm. Measurements were performed with 3-point-bending mode with temperature sweep from −150° C. to 130° C. with a rate of 2° C./min. An oscillation frequency of 1 Hz with an oscillation strain of 0.05% was applied and used initial force was 0.01 N.

Wide Angle X-Ray Scattering (WAXS) Procedure. Analysis of the crystalline structure of the materials was performed using WAXS measurements by means of a computer-controlled goniometer coupled to a sealed-tube source of Cu Kα radiation (Philips), operating at 50 kV and 30 mA. The Cu Kα line was filtered using electronic filtering and the usual thin Ni filter.

Small Angle X-Ray Scattering (SAXS) Procedure. The Kiessig-type camera with sample detector distance of 1.2 m was coupled to an X-ray CuK a low divergence microsource, operating at 50 kV and 1 mA (GeniX Cu-LD by Xenocs, France). The scattering produced by the sample was recorded with the Pilatus 100 K solid-state area detector of the resolution of 172×172 μm2 (Dectris, Switzerland). Dimension of scattering objects was determined from one dimensional sections of 2-D pattern. Background and Lorentz corrections were applied to the curves. Dimension of scattering objects was then calculated from position of the maximum of corrected curves using the Braggs law.

HT-SEC Procedure. The molecular weight and dispersity were determined by means of high temperature size exclusion chromatography (HT-SEC) performed at 150° C. in a HT-SEC-IR instrument equipped with IR⁴ detector (PolymerChar, Valencia, Spain). Three Polymer Laboratories 13 μm PLgel Olexis columns constitute the set. 1,2-dichlorobenzene (o-DCB) was purchased from VWR and used as a eluent at flow rate of 1 mL*min⁻¹. Molecular weight and corresponding dispersities were calculated from HT-SEC analysis with respect to narrow polystyrene standards (PSS, Mainz, Germany).

Scanning Electron Microscopy (SEM) micrographs were obtained with PhenomPro Apparatus equipped with cold cathode field-emission source at an acceleration voltage of 5 kV. Prior to analysis samples were cryogenically fractured and glued on a SEM stub with conducting carbon tape. All samples were then sputter coated with approximately 10 nm layer of gold using Quorum Q150V Plus sputter coater.

### Example 1

### Polyolefin like Polyester Polymers

A diol (14) having a mol. wt. of 6,300 g/mol. was synthesized. The diol had a hydrocarbon backbone containing —CH₂CH₃ branches. The diol can be represented with the following formula, where x and y are mole fractions and have a ratio of 97:3.

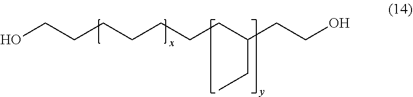

The diol (14) (2.727 mmol) was polymerized, via esterification and polycondensation, with succinic acid (2.99 mmol) using titanium tetra-isopropoxide (1.34 wt. % of the polymer). The esterification was carried out at 190° C. for a period of 2.5 h under nitrogen atmosphere followed by polycondensation for 5 h at 220° C. at 0.05 mbarg. The polymer (15) obtained shows polyolefin like properties. a in formula (15) is an integer denotes number of repeat units.

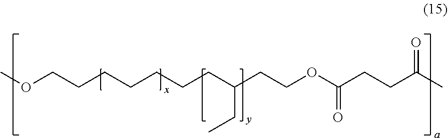

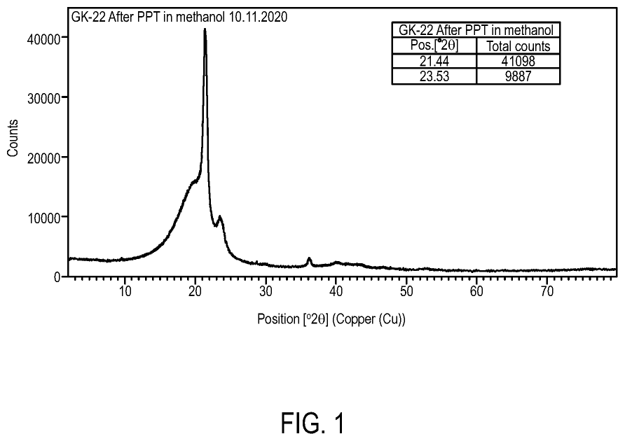

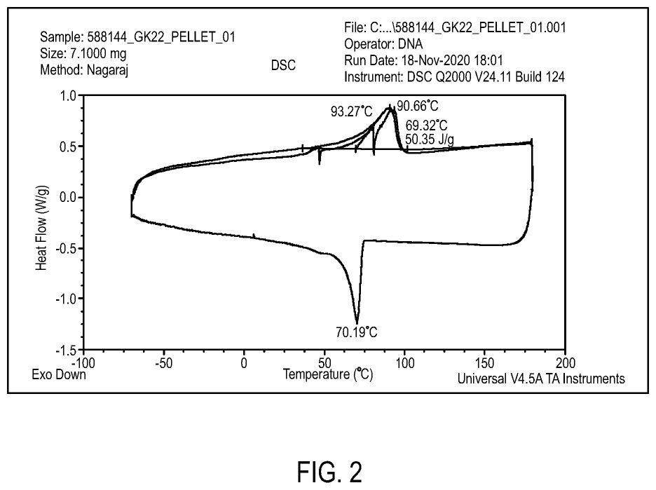

XRD (FIG. 1) of the polymer (15) shows, the polymer is highly crystalline ˜60% and has crystallinity similar to that of conventional LLDPE. Tₘ of the polymer was ˜90.2° C., as measured by differential scanning calorimetry (DSC), FIG. 2. The weight average molecular weight (M_{w}) of the polymer was 106,901-111,217 g/mol, as measured by GPC.

### Example 2

### Depolymerization of Polymer 15 and Repolymerization of the Diol 14

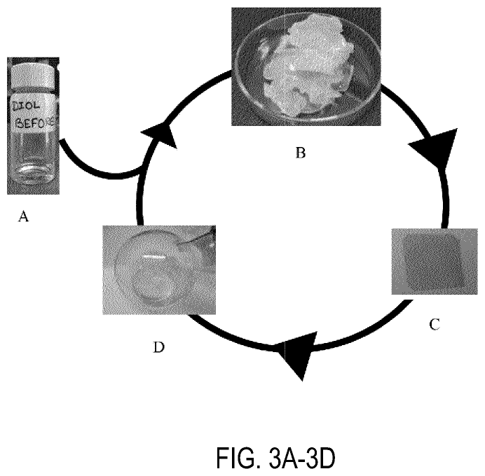

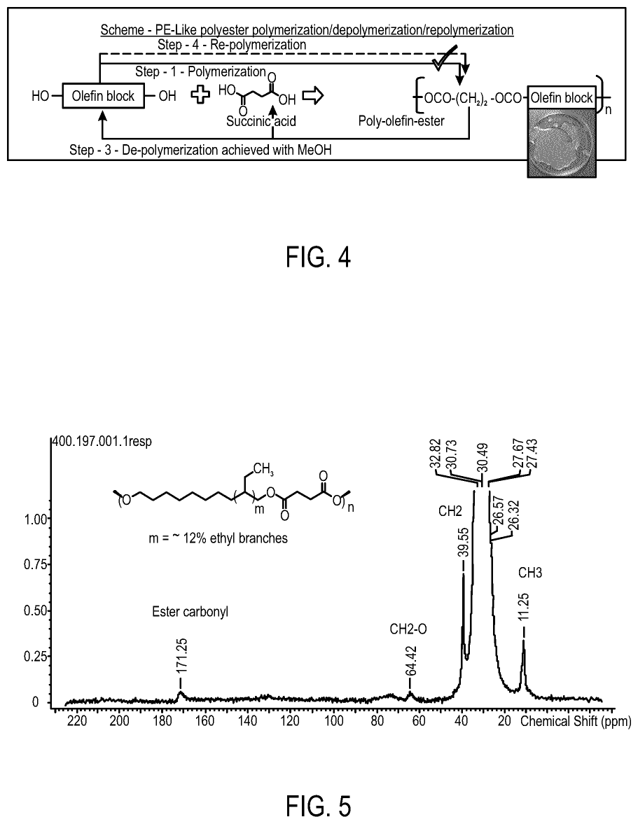

The polymer (15) from the film (FIG. 3C) was depolymerized via methanolysis of the polymer in presence of methanol. Depolymerization was performed at 200° C., 30 barg pressure and for 3 hrs, to obtain the diol (14) and dimethyl succinate. The recycled diol obtained, was repolymerized with succinic acid to obtain the polymer (15). FIG. 3, shows, polymerization depolymerization cycle obtained with the diol (14) and the polymer (15). FIG. 3A shows the starting diol, FIG. 3B shows the polymer (15) obtained by polymerization of the diol (14) with succinic acid. FIG. 3C shows a film or sheet made with the polymer (15). FIG. 3D shows the diol (14) obtained after depolymerization of the polymer (15) from the film. FIG. 4 shows a schematic of polymerization, depolymerization and repolymerization according to one example of the present invention.

### Example 3

### Synthesis of Unsaturated Branched Diol

A multistep synthesis was performed to produce an unsaturated branched polybutadiene diol of the invention. Prior to polymerization, all the glassware were carefully oven dried and charged with argon. All experiments were performed in an inert controlled atmosphere.

Step 1. Synthesis of a hydroxy end group on polybutadiene is shown in (Scheme I). 1,3-Butadiene solution (13.33 g, 36.97 mmol of 15 wt % solution in n-hexane) was added to a reactor under argon atmosphere. Then t-BDMSOPrLi solution (1 mL, 0.5 mmol of 0.5 mol/L see above for analysis method) was added to the reaction mixture under stirring. After complete addition, the reaction mixture was heated to 50° C. and stirred at this temperature for 5 hours. After 5 hours, the reaction mixture was cooled to room temperature and ethylene oxide (15.6 mL, 12.5 mmol of 0.8 mol/L in hexane) was added and allowed the reaction mixture to stir for another 2 h at room temperature. Finally, the reaction mixture was terminated by the addition of degassed (degassing done by freeze-pump-thaw method) methanol (1.5 mL) to form hydroxy end group in polybutadiene. The solution was concentrated and precipitated into an excess of methanol to obtain polybutadiene with one hydroxy end group as a white viscous liquid.

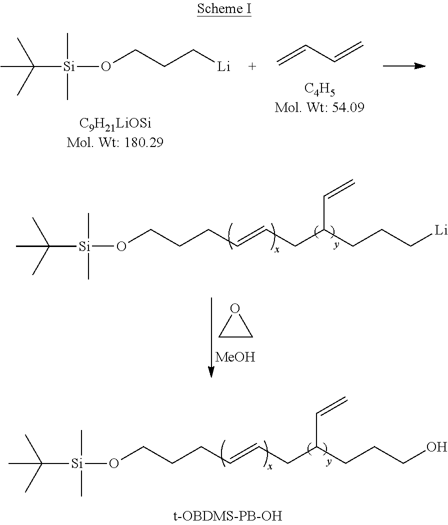

Step 2: Synthesis of dihydroxy terminated polybutadiene is shown in Scheme II. The polybutadiene (1 g) made in Step 1 with one hydroxy end group) was dissolved in THF (10 mL). Subsequently, excess tetrabutylammonium fluoride (TBAF, 1 M in THF); was added to the solution ([TBAF]/[TBDMS] 3:1 weight ratio) at room temperature under stirring and allowed to react for 24 h to obtain the hydroxyl groups at both ends of the polybutadiene. Finally, the polymer was precipitated in methanol and residual solvent was evaporated. The crude product was dissolved in 50 mL of suitable solvent (according to its solubility, either in hexane/cyclohexane/dichloromethane) and washed with water (2×50 mL) to remove any salts present in the crude mixture. The solvent was dried over anhydrous sodium sulfate (˜10 g), filtered and the solvent was evaporated using a rotary evaporator to produce unsaturated OH—PB—OH.

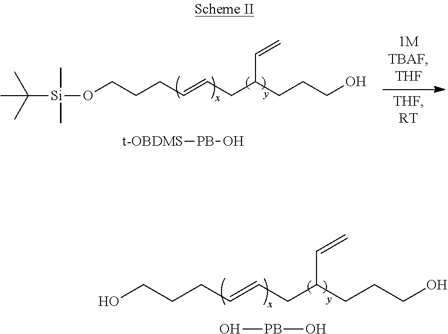

Step 3: Hydrogenation of unsaturated OH—PB—OH is shown in Scheme III. In a 600 mL Parr vessel, transfer/weigh unsaturated OH—PB—OH (24 gm M_{w}-5500) into a conical flask and add cyclohexane (150 ml) into the conical flask. Mix the contents in the conical flask thoroughly and then transfer the contents into the Parr vessel. Add additional cyclohexane (150 ml) into the conical flask. Mix the contents in the conical flask thoroughly and then transfer the contents into the Parr vessel. (Ensure that no reactant is present in the conical flask). Added Pd/CaCO₃ (2.4 g of 3 wt. %) catalyst directly into the Parr vessel. The Parr vessel was sealed and heated to 75° C. at 60 barg (0.6 MPa) until the saturated OH—PB—OH was hydrogenated to greater than 99.5% to form saturated OH—PB—OH. The diol as prepared had a molecular weight of Mₙ=5500 and a degree of branching (12%; C₂) which is within the range (0.0001-12). The degree of hydrogenation>·99.5% (within the range>97%), and the carbon chain length for branching C₂ was within the range (C₁-C₁₂).

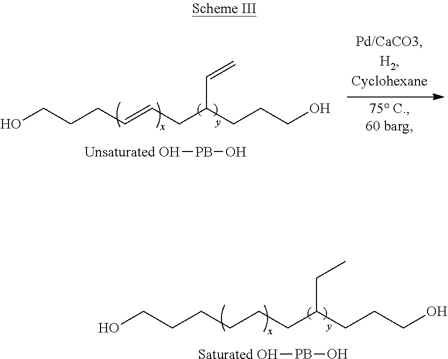

### Example 4

### Synthesis of the LLDE-Mimic Inventive Polymer of the Present Invention, MW=5500

Reaction of (α,ω-dihydroxyl hydrogenated polybutadiene, having ˜12% branching, Example 1) with succinic acid to form the polymer of the present invention is shown in Scheme IV. α,ω-Dihydroxyl hydrogenated polybutadiene (3.6 mmol, Example 1), succinic acid (3.6 mmol, Aldrich) and titanium tetra-isopropoxide (0.12 gms, 1 wt. % of polymer) were introduced into the reactor and the reactor was then heated to 190° C. under stirring and in the presence of a nitrogen atmosphere. The reaction was held for 2.5 hrs at atmospheric pressure to allow esterification to proceed. After that, polycondensation was started by turning off the nitrogen and by gradually reducing the pressure down to ˜0.05 mbar and the temperature was raised to 220° C. The reaction was held for 6 hrs until polycondensation was complete; the vacuum was released by bleeding in nitrogen and the polymer was collected. MW of the resulting inventive polymer was 5500 g/mol. The resulting polymer of the present invention (LLDPE mimic) was characterized by solid state (SS) NMR, DSC, TGA, and XRD. FIG. 5 shows the SSNMR. DSC data of the LLDPE mimic showed a Tₘ and T_{c} of 94° C., 82° C. respectively. TGA in N₂ atmosphere was found to be 460° C. crystallinity of LLDPE mimic was found to be 66% with peaks characteristic of polyethylene at 2θ≈21.7° and 2θ≈23.8° due to (110) and (200) reflections and a weak/broad shoulder band at≈19° representing a semi-amorphous phase as determined by XRD. The resulting polymer had an aliphatic group of 472 carbon atoms (at least 45, preferably 100 to 700), a degree of saturation of 98%, and a melt temperature of 94.6° C. (within the range of (Tₘ) of 40° C. to 180° C.).

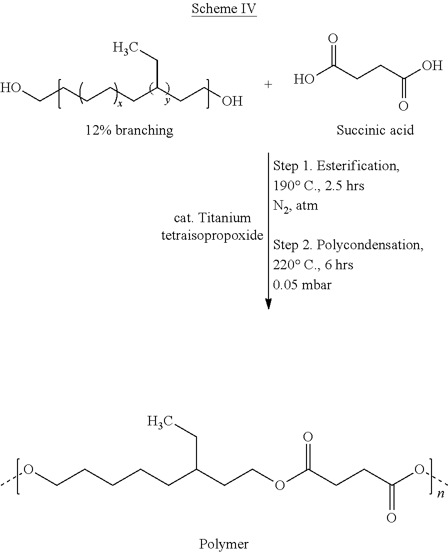

### Example 5

### Synthesis of the LLDPE-Mimic Inventive Polymer of the Present Invention, MW=8500

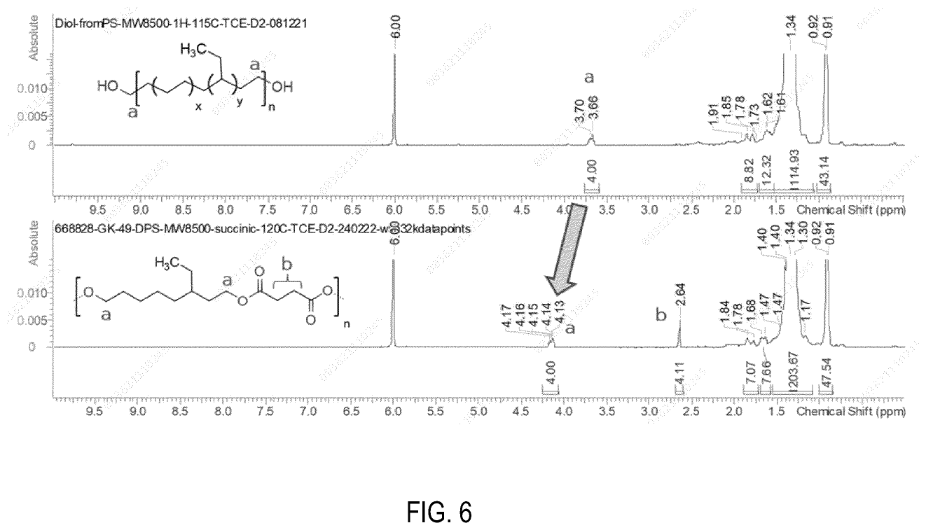

α,ω-Dihydroxyl hydrogenated polybutadiene (Mw=8500 having ˜12% branching, hydrogenation>99.5%) made using the process described in Example 1 was reacted with succinic acid to produce a polymer of the present invention. Inventors, the procedure provided to make the MW=8500 had same amounts and reaction time and temperatures as Example 1. Using the procedure of Example 3, α,ω-Dihydroxyl hydrogenated polybutadiene (12.0 g, 3.6 mmol), succinic acid (0.43 g, 3.6 mmol) and titanium tetra-isopropoxide (0.12 gms) were reacted for 8.5 hours (2.5 hours for esterification and 6 hours for polycondensation) to produce a polymer of the present invention. The inventive polymer was characterized by 1H-NMR (FIG. 6), DSC, TGA and XRD. FIG. 6 shows the ¹H NMR (top) by ¹H NMR in TCE-d2 at 120° C. and compared with α,ω-dihydroxyl hydrogenated polybutadiene (bottom). DSC data of the inventive polymer (LLDPE mimic) showed a Tₘ and T_{c} of 94.6° C. and 77.6° C. respectively. TGA in N₂ atmosphere was found to be 457° C. XRD showed a % crystallinity of 62.7% with peaks characteristic of polyethylene at 2θ≈21.7° and 2θ≈23.8° due to (110) and (200) reflections and a weak/broad shoulder band at ≈19° representing a semi-amorphous phase.

The polymer of this Example had the following characteristics: 1). ester to thousand methylene unit ratio of the polymer was 4.2 within the range of 0.0001 to 40; 2). ethylene branching which is 2 carbon atoms branch (within the range (C₁-C₁₂); 3). degree of branching of ˜65 mol % (desired range 0-12 mol %); and 4). degree of hydrogenation>˜99.5% (within the range>97%), thus making a good example of LLDPE-mimic material. Furthermore, from the results it was determined that polymer of Example 3 had an aliphatic group of 369 carbon atoms (at least 45, preferably 100 to 700), a degree of saturation of 98%, and a melt temperature of 94.6 C (within the range of (Tm) of 40° C. to 180° C.).

### Example 6

### Blend of Inventive Polymer (LLDPE-Mimic) with Polyolefin

The polymer of the present invention (LLDPE-mimic material) was blended with commercially available LLDPE in order to prove miscibility of polyolefin and polyolefin-mimic. This feature allows of blend demonstrate LLDPE properties.

Blend Preparation. Polymer pellets of inventive polymer (1.6 g, Example 3, MW 8.5 kg*mol⁻¹) and SABIC LLDPE 118NJ (6.4 g, MW 125.2 kg*mol⁻¹) were combined and fed into a Xplore MC 15HT twin-screw microcompounder at 180° C. with a flow of nitrogen and total residence time of 5 minutes at 100 RPM. Then, the material was extruded and air cooled. The obtained blend was pressed using LabEcon Series Fontijne Press at 180° C. for 5 minutes to create a film. 15° C./min cooling rate and 100 kN force was applied. Obtained films were conditioned for at least 24 hours before measurements.

Preparation of LLDPE and LLDPE-like Films. Pellets of SABIC LLDPE 118NJ and the inventive polymer (Example 3) were consecutively pressed using LabEcon Series Fontijne Press at 180° C. for 5 minutes to create a film. A 15° C./min cooling rate and 100 kN force was applied. Obtained films were conditioned for at least 24 hours before measurements.

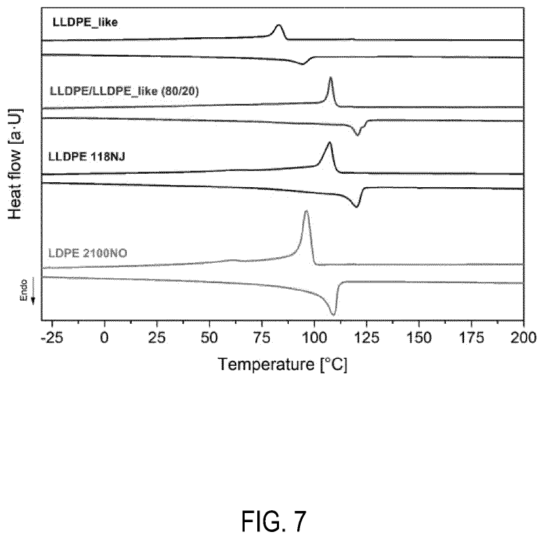

Referring to FIG. 7, DSC thermograms of LLDPE 118NJ, LLDPE-like polyester (inventive polymer) and 80/20 LLDPE/LLDPE-like blend were compared. LDPE 2100NO was used additionally as a reference sample. Melting and crystallization temperature of inventive polymer (LLDPE-mimic polyester) is shifted towards lower temperature comparing to LLDPE. Importantly, the inventive polymer (LLDPE-mimic) displays lower crystallinity degree in comparison with the LLDPE sample. Contrary to the reference samples, the LLDPE/LLDPE-mimic blend reveals non-uniform melting transition. Addition of olefin-mimic component to LLDPE slightly affected the blend's crystallinity degree.

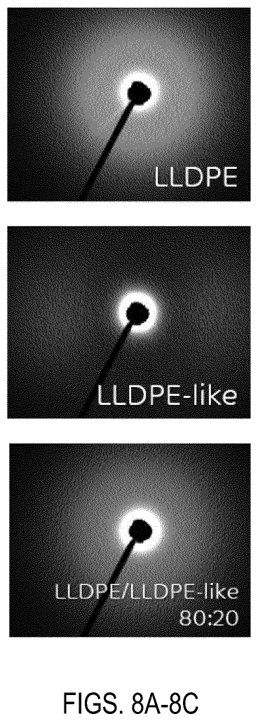

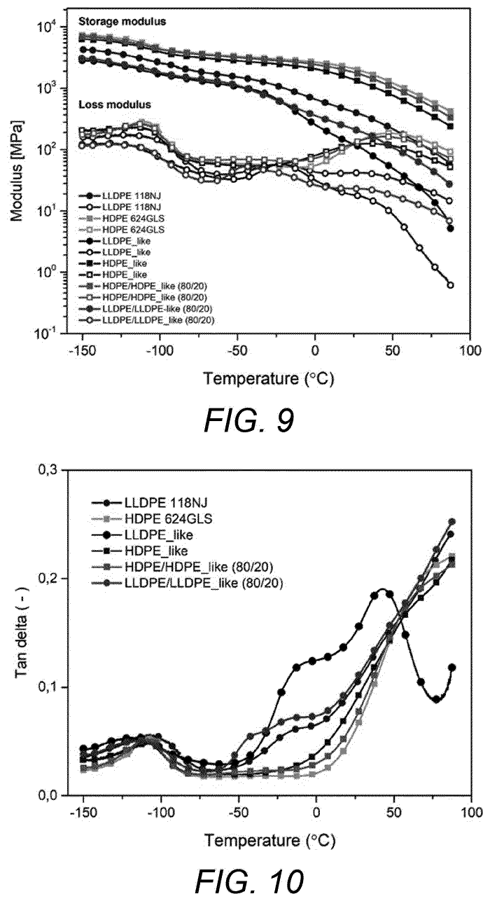

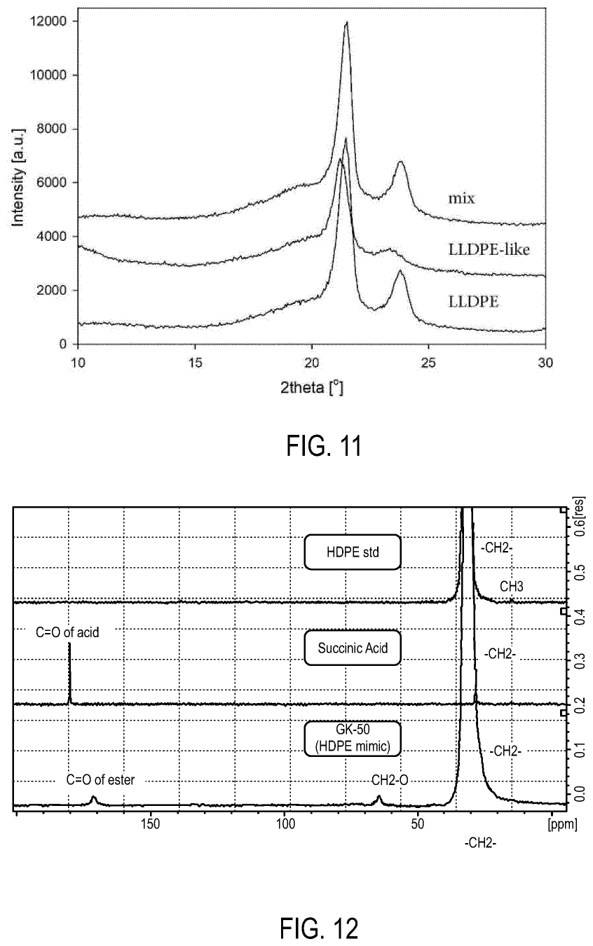

The inventive polymer (LLDPE-mimic) exhibited similar viscoelastic properties to a neat LLDPE in low temperature region. Above 0° C. both storage and loss modulus values drop significantly when compared to the LLDPE reference. LLDPE/inventive polymer (LLDPE-mimic) blend shows similar plot to neat. LLDPE in analyzed temperature range. Miscibility of the PO and PO-like materials is clearly proven by the glass transition temperature analysis of the blends and reference homopolymers as shown in Table 2. The glass transition temperature of the LLDPE, Inventive Polymer (Example 3), and LLDPE/Inventive Polymer blend (Example 4) were −107.2° C., −113.8° C., −110.0° C., respectively. Cocrystallization of the PO and PO mimics The long period values (LP) as determined through X-Ray analysis of the LLDPE, Inventive Polymer (Example 3), and LLDPE/Inventive Polymer blend (Example 4) were 18.7, 12.0, 18.1 respectively. SAXS patterns of LLDPE, LLDPE-like material and corresponding blend are shown in FIGS. 8A-8C. The long period values (LP) are significantly lower for Inventive polymer polyester in comparison to the reference materials. The LP values of the polymer blends are similar to PO. Storage Modulus, Loss Modulus and Loss Factor of polyolefin and polyolefin-like materials are illustrated in FIGS. 9 and 10. FIG. 11 is an illustration of the WAXS profiles of pure LLDPE, LLDPE-like polyester and 80/20 LLDPE/LLDPE-like blend. The orthorhombic unit cell, which is typical for polyethylenes is also recognized for polyolefin-like polyester. For PO mimics (inventive polymer) the signals are slightly shifted towards lower 2θ angle values. Thus, the interplanar distances are respectively larger than in PO. In case of LDPE/LLDPE-mimic blend signals' position is identical as for the neat LLDPE reference sample.

Conclusion. All in all, based on conducted analyses miscibility of LLDPE-mimic (inventive polymer) and commercially available LLDPE is clearly proven. Results from various techniques lead to the same and consistent conclusion that corresponding blend demonstrates thermal, viscoelastic and physical properties of neat LLDPE. Thus, the data showed that the polymer of the present invention was functionally similar to its conventional counterpart.

### Example 7

### Synthesis of Linear Diol MW 3000

Synthesis of α,ω-dihydroxy polyethylene is shown in Scheme VI, where n denotes repeat units. In Step 1. cis-1,4-Diacetoxy-2-butene (2.07 g, 61 12.0 mmol) was added to THF (135 mL) in a two-neck 500 mL Schlenk flask under argon purging. The flask was then transferred to a 35° C. oil bath, and cis-cyclooctene (30 g, 272.2 63 mmol) was added dropwise over 30 min. The addition of a second generation Grubbs catalyst (101.86 mg, 0.12 mmol) solution in THF (3 mL) was started after adding 1 mL cis cyclooctene. After 6 hours of reaction, the mixture was precipitated into acidic methanol (1.2 L with 35% HCl (1.5 g) solution in water (13.5 g). The precipitated polymer, α,ω-diacetoxy terminated polycyclooctene was collected and dried under vacuum for two days.

Step. 2. To convert the end acetoxy groups in α,ω-diacetoxy terminated polycycloocene into hydroxy groups, the polymer was dissolved in THF (137.5 mL) at 40° C. and 25 wt % NaOMe (2.97 g, 55.0 mmol) solution in methanol was added. The solution was stirred for 20 hours and precipitated into methanol (2 72 L) with 35% HCl (1.5 g) solution in water (13.5 g). The isolated α,ω-dihydroxy polycyclooctene was dried under vacuum

Step 3. Hydrogenation of α,ω-dihydroxy polycyclooctene (HO—PCOE—OH): HO—PCOE—OH, (10 g, 90.7 mmol double bonds), p-toluenesulfonyl hydrazide (52.4 g, 281.3 mmol), tributylamine (75.6 mL, 317.6 mmol), butylated hydroxytoluene (50 mg, 0.22 mmol), and o-xylene (385.76 mL) were added to a 1000 mL three-neck round-bottom flask. The mixture was heated to 140° C. and refluxed for 6 hours. After cooling to room temperature, the reaction mixture was poured into methanol. The obtained precipitate was washed with methanol (2×500 mL). The isolated white powder was dried under vacuum the extent of hydrogenation was determined by 1H-NMR and found to be>99%. ¹H-NMR of (TCE-d2, ≥99.5 atom % D, 120° C.): δ: 3.66 (t, CH2—OH, a′); b); 1.61-1.24 (m, —CH₂—). DSC data of α,ω-dihydroxy polyethylene showed a Tₘ and T_{c} of 129° C., 117° C. respectively. TGA in N₂ atmosphere was found to be 452° C.

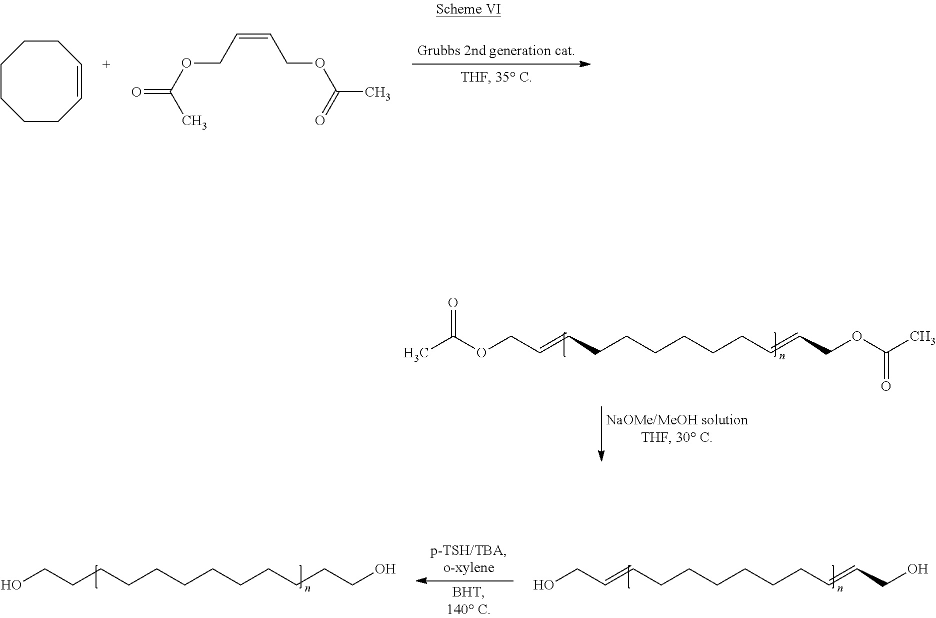

### Example 8

### Preparation of a HDPE-Mimic (Inventive Polymer)

α,ω-dihydroxy polyethylene (12.0 g, 8.2 mmol, Example 5), succinic acid (0.96 g, 8.2 mmol) and titanium tetra-isopropoxide (0.12 gms) were introduced into the reactor and the reactor was then heated to 190° C. under stirring and in the presence of nitrogen atmosphere. The first stage, esterification was carried out for 2.5 hrs at atmospheric pressure and at 190° C. After that, the second stage, polycondensation was started by turning off the nitrogen and by gradually reducing the pressure down to ˜0.05 mbar and the temperature was raised to 220° C. After polycondensation reaction for 3.0 hrs, the vacuum was released by bleeding in the Nitrogen and the polymer was collected. The HDPE mimic (inventive polymer) was characterized by Solid state NMR and it was compared with the standard HDPE (FIG. 12). DSC data of the HDPE mimic (inventive polymer) showed a Tₘ and T_{c} of 119° C., 102° C. respectively.

Results and Discussion. The HDPE-mimic the following key properties: ester to thousand methylene unit ratio for inventive polymer (HDPE-mimic) of this example was 9.4, which is within the range of 0.0001 to 40; which can be classified and demonstrate as HDPE like polymer. Other important characteristics have been within specifications such as branching of ethylene which is 0 carbon atom branch (within the range (C₀-C₁₂) of and degree of branching of ˜0 mol % (see graph below) (within the range (0-12 mol %) and fully hydrogenated >˜99.5% (within the range (>97%) makes it a HDPE mimic materials. The crystallinity of the HDPE-mimic (inventive polymer) was found to be highly crystalline and has a Tₘ of 119° C. The results show that the polymer had an aliphatic group of 212 carbon atoms (at least 45, preferably 100), a degree of saturation of 98%, and a melt temperature of 119° C. (within the range of (Tm) of 40° C. to 180° C.).

### Example 9

### Polymer of the Present Invention (HDPE-MIMIC) Blends

Blend Preparation. Polymer pellets of polymer of the present invention HDPE-like material (1.6 g, Example 8) and SABIC HDPE B6246LS (6.4 g, Commercial grade HDPE) were combined and fed into a Xplore MC 15 HT twin-screw microcompounder at 180° C. with a flow of nitrogen and total residence time of 5 minutes at 100 RPM. Then the material was extruded and air cooled. The obtained blend was pressed using LabEcon Series Fontijne Press at 180° C. for 5 minutes to create a film. 15° C./min cooling rate and 100 kN force was applied. Obtained films were conditioned for at least 24 hours before measurements.

Preparation of HDPE and HDPE-like (inventive polymer) films. Pellets of SABIC HDPE B6246LS and HDPE-like (inventive polymer) material were consecutively pressed pressed using LabEcon Series Fontijne Press at 180° C. for 5 minutes to create a film. A 15° C./min cooling rate and 100 kN force was applied. Obtained films were conditioned for at least 24 hours before measurements.

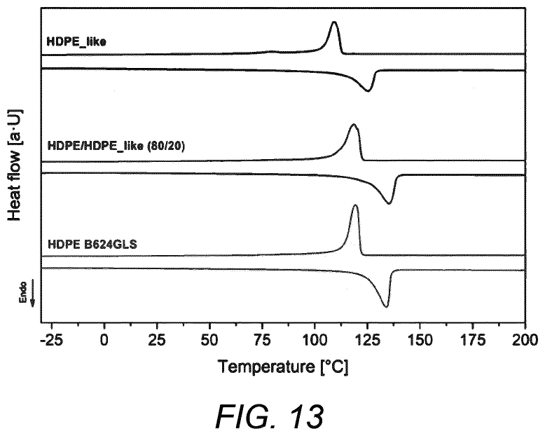

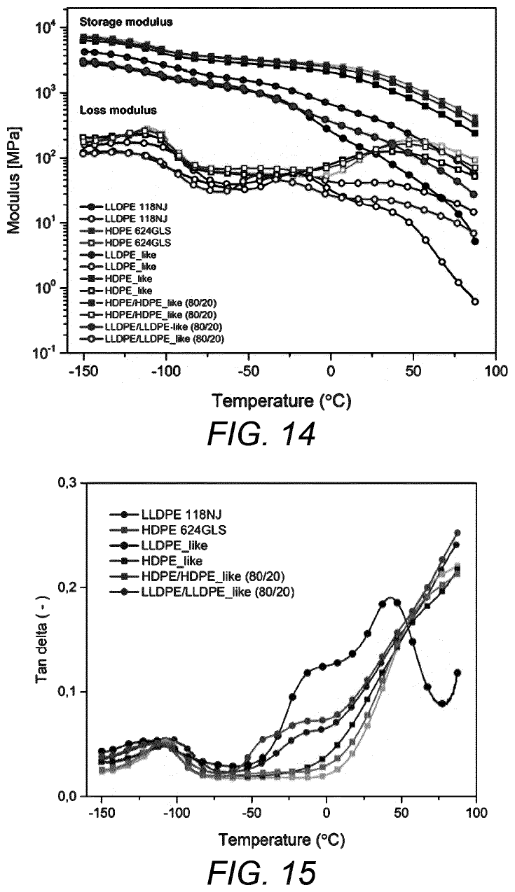

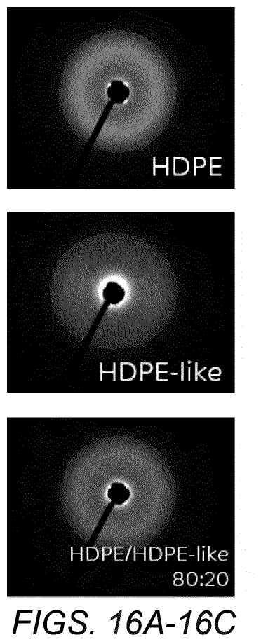

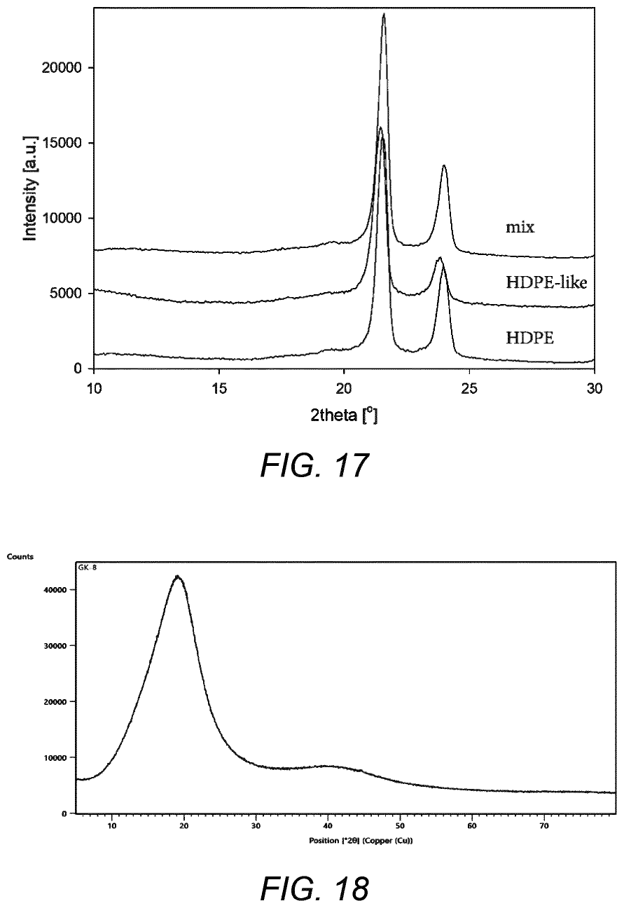

Results and Discussion. The HDPE mimic was characterized by Solid State NMR and it was compared with the standard HDPE reference sample. For HDPE-mimic material Tₘ shows linear relationship with number of methylene units between the ester groups. Tₘ is also dependent on the MW of the final product. Based on DSC analysis it is visible that melting point and crystallization temperature of the HDPE-mimic polyester is lower in comparison to the HDPE reference sample. HDPE-mimic product shows slightly lower crystallinity degree in comparison with the HDPE material. The Tₘ and T_{c} of the blends are similar to a neat HDPE. Mixing HDPE with HDPE-mimic slightly affects the crystalline phase of HDPE. DSC thermograms of HDPE B6246LS, HDPE-mimic polyester and 80/20 HDPE/HDPE-mimic blend are shown in FIG. 13. In terms of viscoelastic properties (FIGS. 14 and 15) HDPE-mimic polyester displayed slightly lower storage and loss modulus than HDPE reference sample. The blend containing 80 wt % of HDPE and 20 wt % of HDPE-mimic (inventive polymer) polyester reveals almost identical viscoelastic properties as HDPE in a wide temperature range. Miscibility of the PO and PO-like materials is clearly proven by the glass transition temperature analysis of the blends and reference homopolymers. The glass transition temperature of the HDPE, Inventive Polymer (Example 8), and HDPE/Inventive Polymer blend (Example 9) were −107.2° C., −110.8° C., and −108.6° C., respectively. Cocrystallization of the PO and PO mimics the long period values (LP) as determined through X-Ray analysis of the HDPE, Inventive Polymer (Example 6), and HDPE/Inventive Polymer blend (Example 7) were 28.2, 24.9, and 27.9 respectively. SAXS patterns of HDPE, HDPE-like material (inventive polymer) and corresponding blend are shown in FIGS. 16A-16C. The long period values (LP) are significantly lower for Inventive polymer polyester in comparison to the reference materials. The LP values of the polymer blends are similar to PO. FIG. 17 is an illustration of the WAXS profiles of pure HDPE, HDPE-like polyester (inventive polymer) and 80/20 HDPE/HDPE-like (inventive polymer) blend. The orthorhombic unit cell, which is typical for polyethylenes is also recognized for polyolefin-like polyester. For PO mimics (inventive polymer) the signals are slightly shifted towards lower 2θ angle values. Thus, the interplanar distances are respectively larger than in PO. In case of HDPE/HDPE-mimic (inventive polymer) blend signals' position is identical as for the neat HDPE reference sample.

Conclusion. All in all, based on conducted analyses miscibility of HDPE-like (PE-Mimic, inventive polymer) and commercially available HDPE is clearly proven. Results from various techniques lead to the same and consistent conclusion that corresponding blend demonstrates thermal, viscoelastic and physical properties of neat HDPE. The data showed that the inventive polymer was functionally similar to its conventional counterpart.

### Comparative Example 1

### Synthesis of a Polymer Using a Commercial Diol

Commercial diol (KRASOL HLBH P 3000, 65% branching, MW=3000, Mn=3100, Cray Valley) was hydrogenated in the same manner as described in step 3 of Example 1 to produce a hydrogenated branched diol (65% branching). The hydrogenated diol was reacted with succinic acid as described in Example 2 with the following amounts of ingredients (hydrogenation diol 20.0 g, 11.07 mmol), succinic acid (1.3 g, 11.07 mmol) and titanium tetra-isopropoxide (0.15 gms). The results show that the polymer had an aliphatic group of 212 carbon atoms (at least 45), a degree of saturation of 98%, and a melt temperature of <40° C. (outside the range of (Tm) of 40° C. to 180° C.) as measured by DSC.

Using the commercial diol (P-3000) the comparative polymer failed to make a PE-Mimic (LLDPE-mimic) as the comparative polymer failed to show any crystallinity. The diol blocks have ethylene (2 carbon atom) substitution (within the range (C₁-C₁₂) of and very high degree of branching ˜65 mol % (see graph below) (outside the range (0-12 mol %) and fully hydrogenated >˜99.5% (within the range (>97%) cannot make it a LLDPE-mimic material.

### Comparative Example 2

### Reaction of Commercial Diol with Sebacic Acid

Hydrogenated commercial diol of Comparative Example 1 (20.0 g, 11.1 mmol,), sebacic acid (2.24 g, 11.1 mmol) and titanium tetra-isopropoxide (0.47 gms) were reacted to form a comparative polymer were introduced into the reactor and the reactor was then heated to 190° C. under stirring and in the presence of nitrogen atmosphere. The first stage, esterification was carried out for 3 hrs at atmospheric pressure. After that, the second stage, polycondensation was started by turning off the nitrogen and by gradually reducing the pressure down to ˜0.05 mbar and increasing the temperature to 220° C. After polycondensation reaction for 4.0 hrs, the vacuum was released by bleeding in nitrogen and the polymer was collected. The resulting polymer did not show any crystallinity (T_{m<40°} C.). Even though the diol blocks have ethylene (2 carbon atom) substitution (within the range (C₁-C₁₂) and were fully hydrogenated >˜99.5% (within the range (>97%), the polymer very high degree of branching ˜65 mol %, which is believed to contributed to the lack of crystallinity. FIG. 18 shows the XRD of the comparative polymer.

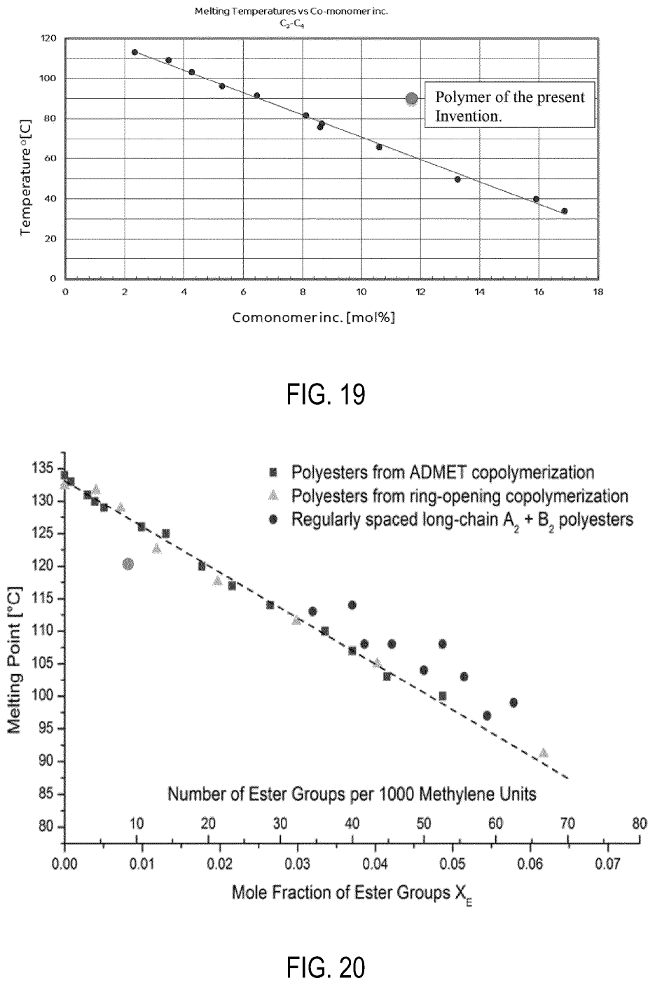

Discussion. Table 1 lists the properties of the inventive diols, the comparative diols, and the polymers produced from the diols. As determined from the data, use of the commercial diol (P-3000) having a high degree of branching failed to produce a PE-Mimic (LLDPE mimic) as they lacked crystallinity (T_{m<40°} C.) as determined by DSC. FIG. 19 illustrates melting temperature vs. mol, % of comonomer (% branching) incorporation of conventional polymers (on the line of the graph) and the polymers of the present invention (circles above the line). As shown, the polymers of the present invention have a higher melting point than the conventional polymers

TABLE 1

| | DIOL | | | | | | | | |
|---|---|---|---|---|---|---|---|---|---|
| | Molecular | | | | | | | | |
| | Weight | | | Branching | Degree of | | | PE-Mimic | |
| | [M_{w}] | | DOB | Carbon | hydrogenation | Diacid | | —COO/1000 | |
| Example | (g/mol) | Funct | (y %) | Length | (%) | (CH₂) | COOH | (CH₂) | T_{M} |
| Example 2 | 5500 | OH | 10 | 2 | 99.5 | 2 | 2 | 5.4 | 94 |
| (Invention) | | | | | | | | | |
| Example 3 | 8500 | OH | 10 | 2 | 99.5 | 2 | 2 | 4.2 | 94.6 |
| (Invention) | | | | | | | | | |
| Comparative | 3000 | OH | 65 | 2 | 97 | 2 | 2 | 9.3 | <40* |
| Example 1 | | | | | | | | | |
| Comparative | 3000 | OH | 65 | 2 | 97 | 2 | 2 | 9.1 | <40* |
| Example 2 | | | | | | | | | |

### Comparative Example 3

### Synthesis of a Comparative Poly(Dodeca Sebacate)

1,12-Dodecane diol (12.0 g, 59.3 mmol, Aldrich), sebacic acid (12.0 g, 59.3 mmol, Aldrich) and titanium tetra-isopropoxide (0.29 gms, Aldrich) were introduced into the reactor and the reactor was then heated to 190° C. under stirring and in the presence of nitrogen atmosphere. The first stage, esterification was carried out for 3.0 hrs at atmospheric pressure. After that, the second stage, polycondensation was started by turning off the nitrogen and by gradually reducing the pressure down to ˜0.05 mbar and the temperature was raised to 220° C. After polycondensation reaction for 4.0 hrs, the vacuum was released by bleeding in the nitrogen and the polymer, poly (dodeca sebacate) was collected. The polymer, poly(dodeca sebacate) was characterized by ¹H-NMR with the following peaks 4.04 (2×CH₂ ester, a); 2.28 (2×CH₂, b); 1.60 (4×CH₂, c), and 1.29 (12×CH₂, d). DSC data of the poly(dodeca sebacate) showed a Tₘ and T_{c} of 83.2° C. and 66.7° C. respectively. Most of the commercial diols when used as feedstock in esterification and condensation reaction to form PE-like polymer does not show PE like properties because it does not meet one important property which is ester to thousand methylene unit ratio for this example 6 (HDPE mimic) was 100 (see table below) which is outside the range of 0.0001 to 40. Due to this high ratio the Tₘ is very low and polymer are soft hence cannot be true PE-mimics.

### Comparative Examples 4 through 10

### Synthesis for Long Chain Aliphatic Polyesters

The linear diol's (1,12-dodecane; 1,8-octane and 1,6-hexane) are purchased from Aldrich which has 12 to 6 (CH₂) in the diol; diacid (sebacic; succinic; tetradecane, dodecane dioic acids) has 2 to 12 (CH₂) which were used in the esterification and condensation reaction to form polymeric materials.

General procedure for long chain aliphatic polyester synthesis (Scheme VII). :α,ω-dihydroxy alkylene (50.0 mmol, Aldrich), α,ω-dicarboxy alkylene (50.0 mmol, Aldrich) and titanium tetra-isopropoxide (1.0 wt % of the polymer, Aldrich) were introduced into the reactor and the reactor was then heated to 190° C. under stirring and in the presence of nitrogen atmosphere. The first stage, esterification was carried out for 3.0 hrs at atmospheric pressure. After that, the second stage, polycondensation was started by turning off the nitrogen and by gradually reducing the pressure down to ˜0.05 mbar and the temperature was raised to 220° C. After polycondensation reaction for 4.0 hrs, the vacuum was released by bleeding in the Nitrogen and the polymer was collected. The polymers were characterized by ¹H-NMR, thermal properties by DSC and crystallinity by XRD and are tabulated in Table 2.

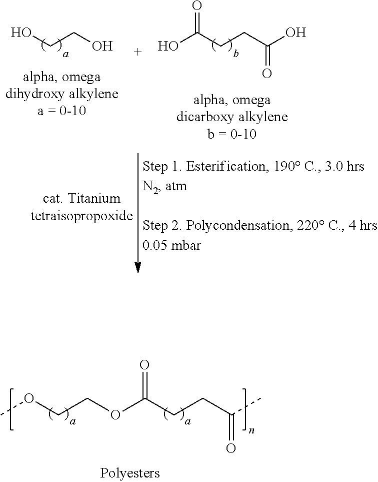

Results and discussion: Most of the commercial diols when used as feedstock in esterification and condensation reaction to form PE-like polymer does not show PE like properties because it does not meet one important property which is ester to thousand methylene unit ratio. The Comparative Examples 4-10 had such ratios ranging from 83 to 167 (Table 2) which is outside the range of 0.0001 to 40. Due to this high ratio the Tₘ is very low and polymers were soft, and hence cannot be true PE-mimics. The results showed that the comparative polymers had an aliphatic group of 6 and 12 carbon atoms (less than 45), a degree of saturation of 98%, and a melt temperature of 65-70° C. (within the range of (Tₘ) of 40° C. to 180° C.). The data showed that the comparative polymers made in accordance with our invention were not functionally similar to its conventional counterpart. FIG. 20 shows melting temperature vs. mole fraction of ester groups X_{E} and number of ester groups per 1000 methylene units for comparative polymers (square and triangle monikers) and the polymers of the present invention (round monikers). As shown, the polymers of the present invention containing less than 40, such as 0.01 to 40 ester groups, per 1,000 backbone carbon atoms, having relatively high degree of saturation, and/or having relatively low degree of branching, have a higher melting point than the comparative polymers with the same number of ester groups.

TABLE 2

| | DIOL | | | | | | | | |
|---|---|---|---|---|---|---|---|---|---|
| | Molecular | | | | | | | | |
| | Weight | | | | Degree of | | | PE-Mimic | |
| | [M_{w}] | | | | hydrogenation | Diacid | | —COO/1000 | T_{M} |
| Example | (g/mol) | Functionality | DOB | BCL | (%) | (CH₂) | COOH | (CH₂) | ° C. |
| 2 (Inventive | 3000 | OH | 0 | 0 | 99.5 | 2 | 2 | 9.4 | 119 |
| polymer) | | | | | | | | | |
| Comparative 4 | 23940 | OH | 0 | 0 | 100 | 8 | 2 | 100 | 83.6 |
| Comparative 5 | 44263 | OH | 0 | 0 | 100 | 2 | 2 | 142.9 | 78.3 |
| Comparative 6 | 136935 | OH | 0 | 0 | 100 | 8 | 2 | 125 | 70.7 |
| Comparative 7 | 26759 | OH | 0 | 0 | 100 | 6 | 2 | 166.7 | 65.4 |
| Comparative 8 | 56479 | OH | 0 | 0 | 100 | 3 | 2 | 133.3 | 71.2 |
| Comparative 9 | 74229 | OH | 0 | 0 | 100 | 12 | 2 | 83.3 | 87.9 |
| Comparative 10 | 60564 | OH | 0 | 0 | 100 | 10 | 2 | 90.9 | 85.7 |

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill, in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

What is claimed is:

1. A polymer comprising repeating units of Formula I: 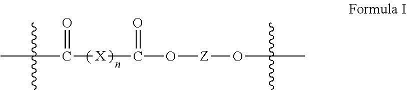
wherein n is 1 and denotes number of repeat units;
X is an aliphatic group; and
Z is an aliphatic hydrocarbon group comprising at least 45 carbon atoms, preferably 45 to 1,000 carbon atoms, and has a degree of saturation of 98 to 100%,
wherein the polymer comprises 0.01 to 40 ester groups per 1000 backbone carbon units, and
wherein the polymer has a melt temperature (Tₘ) of 40° C. to 180° C. as measured by DSC at a heating rate of 10° C. per min,

2. The polymer of claim 1, wherein Z is a linear or branched hydrocarbon having a degree of branching (DB) of 0 to 10%.

3. The polymer of claim 1, wherein Z is a branched hydrocarbon comprising C₁ to C₁₀ hydrocarbon branches.

4. The polymer of claim 1, wherein Z is a polyethylene, poly(ethylene-co-propylene), poly(ethylene-co-1-butene), poly(ethylene-co-1-hexene), or poly(ethylene-co-1-octene) group.

5. The polymer of claim 1, wherein Z is polypropylene group, such as an atactic, isotactic, or syndiotactic polypropylene group.

6. The polymer of claim 1, wherein X comprises 45 to 1,000 carbon atoms, or X is C₁ to C₄₄ aliphatic group, preferably a C₁ to C₂₀ aliphatic group.

7. The polymer of claim 6, wherein X is selected from 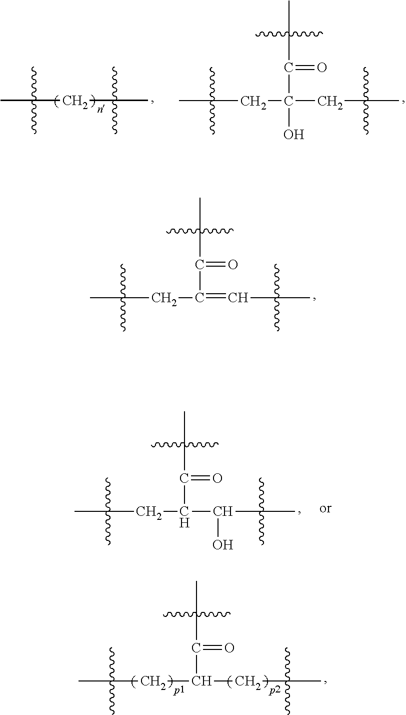
wherein n′ is an integer from 1 to 15, and denotes number of repeat units, and wherein p1 and p2 is independently 1, 2, or 3, and denote number of repeat units.

8. The polymer of claim 1, comprising a number average molecular weight of 10,000 to 1,000,000 g/mol, preferably of 20,000 to 500,000 g/mol, more preferably of 40,000 to 200,000 g/mol, said number average molecular weight being determined as the polyethylene equivalent molecular weight by high temperature size exclusion chromatography performed at 160° C. in trichlorobenzene using polyethylene standards.

9. The polymer of claim 1,
i) comprising repeating units of Formula II, or
ii) comprising repeating units of Formula III, or
iii) comprising repeating units of Formula IV, or
wherein 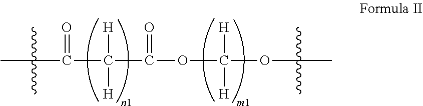
wherein,
n1 is an integer from 0 to 15 and denotes number of repeat units, and
m1 is an integer from 100 to 500 and denotes number of repeat units, 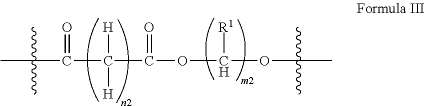
wherein,
n2 is an integer from 0 to 15 and denotes number of repeat units,
m2 is an integer from 100 to 520 and denotes number of repeat units,
R¹ is —H or —CH₂CH₃, and varies independently between H and CH₂CH₃ in the repeating units —CHR¹—, and
—(CHR¹)ₘ₂ group has a DB of 0.1 to 5%, 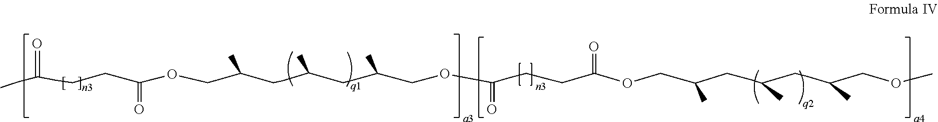
wherein n3 is an integer from 0 to 14 and denotes number of repeat units,
q1 and q2 are independently an integer from 25 to 200 and denote number of repeat units, and
a3 and a4 are independently an integer and denotes number of repeat units.

10. The polymer of claim 1, comprising repeating units of a first unit having the formula of Formula I, and repeating units of a second unit having the formula of Formula I, wherein X of the first unit has a different chemical formula than the X of the second unit.

11. A method for forming the polymer of claim 1, the method comprising:
reacting a α,ω-dihydroxy compound having a formula of HO—Z—OH, with i) an acid having a formula of Formula V, ii) an ester of the acid having the formula of Formula V, and/or iii) a cyclic anhydride of the acid having the formula of Formula V, 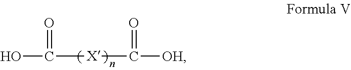
wherein n is 1 and denotes number of repeat units, and
X′ is an aliphatic group, preferably X′ is selected from 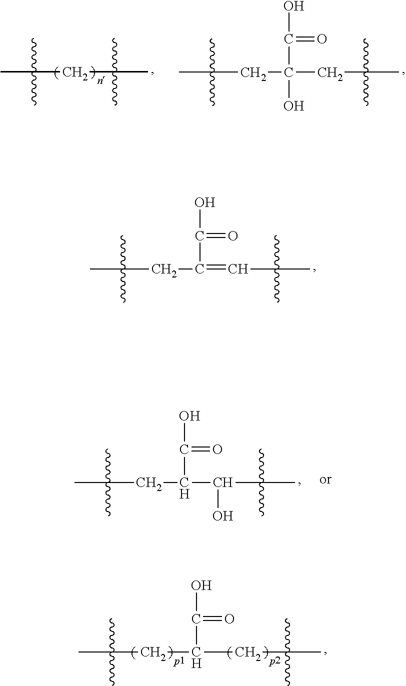
or any combinations thereof,
wherein n′ is an integer from 1 to 15 and denotes number of repeat units, and wherein p1 and p2 are independently 1, 2, or 3, and denote number of repeat units.

12. The method of claim 11, wherein the acid is oxalic acid, malonic acid, succinic acid, maleic acid, glutaric acid, adipic acid, pimelic acid, suberic acid, azelaic acid, sebacic acid, citric acid, aconitic acid, isocytric acid, propane-1,2,3-tricarboxylic acid, or pentane-1,3,5-tricarboxylic acid, or any combinations thereof.

13. The method of claim 11, wherein the ester is methyl, ethyl and/or propyl ester, and/or wherein the cyclic anhydride is malonic anhydride, succinic anhydride, maleic anhydride, glutaric anhydride, adipic anhydride, pimelic anhydride, suberic anhydride, azelaic anhydride, sebacic anhydride or any combinations thereof.

14. The method of claim 11, wherein the α,ω-dihydroxy compound is reacted with the acid or ester or cyclic anhydride thereof at i) a temperature of 90 to 250° C., and/or ii) under inert atmosphere and/or vacuum.

15. A method for recycling a polymer of claim 1, the method comprising contacting the polymer (Formula 1) with water and/or an alcohol under conditions suitable to depolymerize the polymer through hydrolysis and/or alcoholysis to produce a α,ω-dihydroxy compound having a formula of HO—Z—OH, and an acid having a formula of Formula V, and/or an ester thereof,
wherein Formula V is 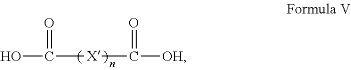
wherein n is 1 and denotes number of repeat units, and
X′ is an aliphatic hydrocarbon group.
